(19) United States
(12) Patent Application Publication
(10) Pub. No.: US 2006/0034791 A1
(43) Pub. Date: Feb. 16, 2006

(54) Cosmetic composition comprising a silicone polymer

(75) Inventors: Momoko Shimizu, Kawasaki-shi (JP); Xavier Blin, Paris (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 11/181,951

(22) Filed: Jul. 15, 2005

Related U.S. Application Data
(60) Provisional application No. 60/591,619, filed on Jul. 28, 2004.

(30) Foreign Application Priority Data
Jul. 16, 2004 (FR) 04 51556

(51) Int. Cl. A61K 8/894

(52) U.S. Cl. 424/70.12

(57) ABSTRACT

The disclosure relates to a make-up and/or care cosmetic composition suitable for application to human skin, lips and/or superficial body growths and exhibiting an improved hold of the color while having a satisfactory or improved gloss, and a satisfactory or improved comfort.

This non-provisional application claims the benefit of French Application No. 04 51556 filed on Jul. 16, 2004 and U.S. Provisional Application No. 60/591,619 filed on Jul. 28, 2004.

### BACKGROUND

Generally, it is desirable for cosmetic compositions to confer an aesthetic effect when applied and to maintain this aesthetic effect over time. It is further desirable for cosmetic compositions to withstand the various external factors capable of modifying their aesthetic effect, such as sweat or tears for a foundation, or saliva for a lipstick.

Cosmetic compositions, such as lipsticks, should not migrate into wrinkles or fine lines and should not transfer onto a fabric. In addition, cosmetic compositions should be pleasant to apply and should maintain a feeling of comfort over time while retaining satisfactory aesthetic properties.

Conventional cosmetic compositions can be obtained by the addition of a volatile oil to the formulation of the composition. The evaporation of the volatile oil after application of the cosmetic composition to skin or lips results in a reduction in the liquid fraction and in the deposited layer becoming concentrated. However, the evaporation of the volatile oil produces a feeling of discomfort and a detrimental change in the gloss effects of the cosmetic composition containing the volatile oil.

Conventional cosmetic compositions can also be obtained from salts of alginic acids that are soluble in water and aqueous dyes, avoiding the use of volatile oil. Examples of such conventional compositions are disclosed in Japanese Patent Application No. JP 5-271034 and European Patent No. EP 0 585 981. However, such compositions exhibit the disadvantage of strongly marking the skin.

Other compositions use non-ionic silicone derivatives, such as polyoxyalkylenated organopolysiloxane derivatives, in order to avoid the use of volatile oil. Examples of such compositions are disclosed in European Patent No. EP 0 548 694 and in Japanese Patent Applications Nos. JP 7-025728, JP 7-330547 and JP 8-027274. Compositions comprising alkylglyceryl ether silicone derivatives have been disclosed in Japanese Patent Applications Nos. JP 6-305933 and JP 7-330547. Compositions with an oily base and comprising polyglycerylated silicone derivatives or fluoroalkylpolyglycerylated silicone derivatives have been provided in Japanese Patent Applications Nos. JP 6-157236, JP 9-071504 and JP 10-310504. Compositions comprising alkylglycerol silicone derivatives have also been disclosed in European Patent No. EP 0 475 130 and in Japanese Patent Applications Nos. JP 2-844453 and JP 2-587797. Other compositions comprising silicone derivatives hydroxylated by saccharides, butylene glycol or glycerol have been disclosed in Japanese Patent Applications Nos. JP 5-186596 and JP 6-145023. However, in the presence of water, these compositions form a gel at the surface of the skin which results in a feeling of discomfort over time.

### SUMMARY

The disclosed cosmetic compounds overcome the disadvantages of conventional cosmetic compositions and exhibit an improved hold on keratinous substances, while maintaining or improving the gloss and/or the comfort.

The disclosure relates to make-up and/or care cosmetic compositions suitable for application to skin, lips and/or superficial body growths. In embodiments, the cosmetic compositions exhibit an improved hold of color while having at least a satisfactory, and in various embodiments improved, gloss and/or comfort. In embodiments, the cosmetic compositions are suitable in lipsticks, lip balms, lip pencils, liquid or solid foundations, casting as sticks or in dishes, concealers and products for coloring the skin, temporary tattoos, and products for making up the eyes, such as eyeliners, eyeliners in the form of pencils, mascaras, mascaras in the form of cakes, and eye shadows.

The term “keratinous substances” encompasses skin, mucous membranes (such as lips), nails, and keratinous fibers (such as eyelashes and hair).

In embodiments, the incorporation of at least one silicone polymer as described below in a cosmetic composition surprisingly improves the hold and/or the comfort of cosmetic compositions while maintaining a good level of gloss. This is all the more surprising as, in the prior art, the improvement in the hold of a composition is made at the expense of the gloss and/or of the comfort.

In embodiments, cosmetic compositions containing a silicone polymer of general formula (I):
R¹ₐR²_{b}R³_{c}SiO_{(4-a-b-c)/2} (I)
in which R¹ is in particular an alkyl radical, R² is represented by the general formula (III):
   Q—O—X (III)
in which Q is a divalent hydrocarbon radical and X is a polyhydroxylated hydrocarbon radical,
and R³ is an organosiloxane group of general formula (IV): 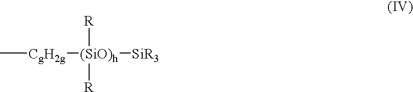
   in which each of the R radicals represents, independently of one another, an alkyl radical, exhibit an improved hold on keratinous substances without harming the gloss and the comfort of the deposited layer.

### DETAILED DESCRIPTION OF EMBODIMENTS

The hold, the gloss and the comfort of a deposited layer of the disclosed cosmetic compositions can be evaluated according to any method known to a person skilled in the art.

Hold of the Composition

The term “hold” is understood to mean the property of a cosmetic composition to keep from transferring onto objects with which the compositions may come into contact. The term “hold” is also understood to mean the property of a cosmetic composition to withstand interaction with liquids, such as tears or sweat, and to withstand contact with foods during a meal. The term “hold” is also understood to mean the property of a cosmetic composition to keep from migrating during the initial tracing of the make-up, such as the initial tracing of lipsticks, into the wrinkles and fine lines of the contour of the lips.

Transfer of cosmetic compositions necessitates regular renewal of the application of the cosmetic composition. The cosmetic composition, once applied to skin, lips and/or superficial body growths, may be detrimentally affected during contact with liquids (for example during contact with water or drinks consumed during a meal), and may be detrimentally affected during contact with oils (such as during contact with food oils, sebum, and/or saliva). The hold of the color of a cosmetic composition can be characterized by the hold of its color towards water and/or towards oil.

In embodiments, the cosmetic compositions do not transfer, at least in part, to objects with which the composition comes into contact. In embodiments, the cosmetic compositions leave only lessened marks on substrates with which the compositions contact, such as on a glass, a cup, a cigarette, a handkerchief, an item of clothing, and/or skin, as compared to the marks left by conventional cosmetic compositions. In embodiments, the disclosed cosmetic compositions exhibit good hold of color.

The hold properties of embodiments of the cosmetic compositions can be characterized by the measurement of at least one of the following four parameters: the absence of transfer, the hold of the color towards water, the hold of the color towards oil, and the absence of migration. The parameters of absence of transfer, of hold of the color towards water, and of hold of the color towards oil can be measured one after the other according to the protocol described below.

The measurements can be carried out, for example, on the inner face of a forearm that has been washed and allowed to dry naturally at ambient temperature for 5 minutes. In embodiments, the cosmetic composition to be tested, for example a lipstick, can be applied to three regions of the inner face of the forearm. In embodiments, the skin surface on which the measurements are carried out is greater than approximately 1 cm². In embodiments, the measurements are carried out on circular regions with a diameter of approximately 3 cm.

Approximately the same amount of cosmetic composition is applied to each of the three regions. In embodiments, this can be confirmed by measuring the weight of the cosmetic composition, for example the lipstick, after each of the applications, or by preparing in advance equivalent amounts of cosmetic composition to be tested. In embodiments, an amount of approximately 2 mg is used for an area of approximately 1 cm². In embodiments, an amount of approximately 28 mg is used for an area that has a diameter of approximately 3 cm.

After application of the cosmetic composition, the initial color (L₁*a₁*b₁*) of each of the three areas is measured and the mean value obtained corresponds to the initial color of the composition. In embodiments, the color of the cosmetic compositions tested can be measured with a Minolta calorimeter of the CR200 or CR300 or CM500 or CM1000 or CM2000 series. In embodiments, the color of the cosmetic compositions tested can be measured with a Minolta colorimeter of the CR200 series

Next, in embodiments, 20 mg/cm² of water is applied to each of the regions to be tested. In embodiments, approximately 280 mg of water are applied for areas having a diameter of approximately 3 cm. Each of the regions to be tested is subsequently subjected to manual massaging for a few seconds. In embodiments, each of the regions to be tested is manual massaged for approximately 2 to approximately 5 seconds. In embodiments, each of the regions to be tested is manual massaged for approximately 2 seconds.

In embodiments, the initial color (L₀*a₀*b₀*) of a commercial white paper handkerchief, such as Kleenex handkerchief, is measured, and the handkerchief is applied to each test region for approximately 5 seconds at a force of approximately 100 g/f. In embodiments, the force can be applied with a DPZ-5N digital force gauge from the manufacturer Imada Co. Ltd.

A transfer value T is obtained by subtracting the color of the handkerchief measured before application to the area to be tested (L₀*a₀*b₀*) from the mean color (L₂*a₂*b₂*) corresponding to the mean of the color values obtained for each handkerchief after their application to each test area.

The difference in color ΔE(T) between the color of the handkerchief before and after its application to the test areas is then determined:
ΔE(T)=\sqrt{(L₂*−L₀*)²+(a₂*−a₀*)²+(b₂*−b₀*)²}
The lower the value ΔE (T) obtained, the more the cosmetic composition is regarded as exhibiting a good level of absence of transfer.

In embodiments, the cosmetic compositions have a transfer value ΔE (T) of between 0 and approximately 45. In embodiments, the cosmetic compositions have a transfer value ΔE (T) of less than or equal to about 45. In embodiments, the cosmetic compositions have a transfer value ΔE (T) of less than or equal to about 40. In embodiments, the disclosed cosmetic compositions have a transfer value ΔE (T) of less than or equal to about 35.

The mean color of the composition after application of the handkerchief (L₃*a₃*b₃*) is subsequently measured.

In embodiments, a cosmetic composition's hold of the color towards water can be obtained after carrying out the transfer test. The hold of the color towards water (W) is equal to the difference in color between the mean initial color of the cosmetic composition applied to the forearm (L₁*a₁*b₁*) and the mean color of the region of the forearm carrying the cosmetic composition after application of the water and of the handkerchief (L₃*a₃*b₃*):
ΔE(W)=\sqrt{(L₃*−L₁*)²+(a₃*−a₁*)²+(b₃*−b₁*)²}
The lower the value obtained, the more the cosmetic composition is regarded as having a good hold of the color towards water.

In embodiments, the cosmetic compositions have a hold of the color of from 0 to approximately 15. In embodiments, the cosmetic compositions have a hold of the color towards water of less than or equal to approximately 15. In embodiments, the cosmetic compositions have a hold of the color towards water of less than or equal to approximately 10. In embodiments, the cosmetic compositions have a hold of the color towards water of less than or equal to approximately 6.

In embodiments, the hold of the color towards oil of the cosmetic compositions is tested by applying approximately 20 mg/cm² of food oil (for example, rapeseed oil, soybean oil, or sunflower oil) to each test area of the forearm, followed by manual massaging for a few seconds. In embodiments, each test area is massaged for approximately 2 to approximately 5 seconds. In embodiments, each test area is massaged for approximately 2 seconds. In embodiments, a commercial white paper handkerchief, such as a Kleenex handkerchief, is subsequently applied to the test region for approximately 5 seconds at a force of approximately 100 g/f. In embodiments, the force can be applied with a DPZ-5N digital force gauge from the manufacturer Imada Co. Ltd.

In embodiments, a cosmetic composition's hold of the color towards oil (O) is equal to the difference between the mean color of the cosmetic composition remaining on the forearm after the massaging with oil and after the application of the handkerchief (L₄*a₄*b₄*) and the initial mean color (L₁*a₁*b₁*):
ΔE(O)=\sqrt{(L₄*−L₁*)²+(a₄*−a₁*)²+(b₄*−b₁*)²}
Determining a cosmetic composition's hold of the color towards oil makes it possible to evaluate the maintenance of a cosmetic composition, such as a lipstick, during a meal.

In embodiments, the cosmetic compositions may contain a silicone polymer in a sufficient amount such that the hold of the color towards oil of a deposited layer of the cosmetic composition, once spread over a substrate, is less than or equal to approximately 25. In embodiments, the cosmetic compositions may contain a silicone polymer in a sufficient amount such that the hold of the color towards oil is less than or equal to approximately 10. In embodiments, the cosmetic compositions may contain a silicone polymer in a sufficient amount such that the hold of the color towards oil is less than or equal to approximately 8.

In embodiments, the cosmetic compositions may contain a silicone polymer in a sufficient amount such that the transfer value of the cosmetic composition, once spread over a substrate, is less than or equal to approximately 35. In embodiments, the cosmetic compositions may have a hold of the color towards water of less than approximately 6 and a hold of the color towards oil of less than or equal to approximately 8.

Mean Gloss of the Composition

The term “mean gloss” denotes the gloss as can be measured conventionally using a glossmeter. For example, a glossmeter of Minolta GM268 type can be used. In embodiments, measurements are carried out on test regions with an area of greater than approximately 1 cm². In embodiments, the areas over which the cosmetic compositions to be tested are spread are approximately 2.5 cm×4 cm.

In embodiments, the cosmetic compositions to be tested are applied to a Bioskin-type synthetic surface. In embodiments, the amount of the cosmetic compositions to be applied is approximately 1 mg/cm². The value of the gloss is obtained by measuring the reflectance at an angle of approximately 60°. In embodiments, five measurements per sample are taken, the measurements exhibiting the highest and lowest values are discarded, and a mean is produced using the three remaining measurements.

In embodiments, the cosmetic compositions contain a silicone polymer of general formula (I) in a sufficient amount such that the mean gloss at 60° of a deposited layer of the cosmetic composition, once spread over a substrate, is equal to or greater than approximately 30 over 100. In embodiments, the disclosed cosmetic compositions contain a silicone polymer of general formula (I) in a sufficient amount such that the mean gloss is equal to or greater than approximately 30. In embodiments, the disclosed cosmetic compositions contain a silicone polymer of general formula (I) in a sufficient amount such that the mean gloss is equal to or greater than approximately 40. In embodiments, the disclosed cosmetic compositions contain a silicone polymer of general formula (I) in a sufficient amount such that the mean gloss is equal to or greater than approximately 45.

Comfort

In embodiments, the comfort of a cosmetic composition is evaluated using a tensile test. Using a strip of latex, this test predicts the ability of a deposited layer of the cosmetic composition to withstand the flaking and peeling that can result from movements of the skin.

In embodiments, samples of a cosmetic composition are applied to test areas of a strip of latex, for example areas of 2.54×2.54 cm. In embodiments, a strip of latex with a width of 2.54 cm is obtained by cutting out from the region of the wrist of a glove, for example, of the type Ansell Edmond Industrial Technicians Ref. #390, size 9.

In embodiments, the cosmetic composition is applied to the strip of latex using a disposable lip brush, for example of the type produced by Femme Cosmetics Inc., LA. In embodiments, the amount of cosmetic composition to be deposited is such that the weight of dry material of the composition should be approximately 20 mg. In embodiments, the weight of the strip of latex devoid of cosmetic composition (A) is subtracted from the weight of the strip of latex comprising the deposited layer of cosmetic composition (B). In embodiments, this difference corresponds to the weight of dry film and is approximately 20±2 mg. In embodiments, the samples thus prepared are left at ambient temperature for 24 hours.

The strip of latex carrying the test samples is subsequently drawn in such a way that the region supporting the test samples reaches a length of, for example, approximately 1.75 inches (4.445 cm). The fragments of film of the cosmetic composition detached on the strip of latex are observed and then removed, for example, by sweeping with the lip brush. The weight of the strip of latex comprising the remaining cosmetic composition (D) is subsequently measured. The percentage loss in weight of the film of the cosmetic composition is calculated using the following equation:
Index of Comfort=[(D−A)/(B−A)]×100.
In embodiments, the measurements are repeated three times for each cosmetic composition tested. In embodiments, the Index of Comfort of the disclosed cosmetic composition is equal to the mean of three such measurements.

In embodiments, the disclosed cosmetic compositions contain a silicone polymer of general formula (I) in a sufficient amount such that the Index of Comfort of a deposited layer of the cosmetic compositions, once spread over a substrate, is equal to or greater than approximately 90 over 100. In embodiments, the disclosed cosmetic compositions contain a silicone polymer of general formula (I) in a sufficient amount such that the Index of Comfort is equal to or greater than approximately 95 over 100.

In embodiments, the disclosed cosmetic compounds contain a combination of a silicone polymer of formula (I) and of a silicone oil, resulting in improved cosmetic qualities, such as improved gloss and/or hold properties.

A first embodiment relates to anhydrous cosmetic compositions comprising, in a physiologically acceptable medium, at least one silicone polymer of general formula (I):
R¹ₐR²_{b}R³_{c}SiO_{(4-a-b-c)/2} (I)
in which:
a, b and c are such that a varies from 1 to 2.5 and b and c, independently of one another, vary from 0.001 to 1.5,
R¹, identical or different, is chosen from:
   C₁-C₃₀ alkyl radicals, optionally substituted by one or more fluorine atoms or amino and/or carboxyl groups,
   aryl or aralkyl radicals, and
   radicals of general formula (II):
      —C_{d}H_{2d}—O—(C₂H₄O)ₑ(C₃H₆O)_{f}R⁴ (II)
   with:
      R⁴ being a C₁-C₃₀ hydrocarbon radical or an R⁵—(CO)— radical with R⁵ being a C₁-C₃₀ hydrocarbon radical, and
      d, e and f being integers such that d varies from 0 to 15 and e and f, independently of one another, vary from 0 to 50,
   and combinations thereof,
R² is a radical represented by the general formula (III):
   -Q—O—X (III)
with:
   Q being a divalent C₂-C₂₀ hydrocarbon radical which can include at least one ether bond and/or at least one ester bond, and
   X being a polyhydroxylated hydrocarbon radical,
R³ is an organosiloxane of general formula (IV): 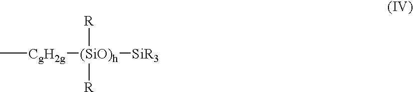
with:
   each of the R radicals representing a C₁-C₃₀ alkyl radical, optionally substituted by one or more fluorine atoms, or aryl or aralkyl radicals,
   g and h being integers such that g varies from 1 to 5 and h varies from 0 to 500,
      and at least one silicone oil.

A second embodiment relates to anhydrous cosmetic compositions comprising in a physiologically acceptable medium, at least one silicone polymer of general formula (I) as defined above, the polymer contained in a sufficient amount in the cosmetic compositions such that the transfer value of a deposited layer of the cosmetic compositions, once spread over a substrate, is less than or equal to approximately 45.

A third embodiment relates to anhydrous cosmetic compositions comprising, in a physiologically acceptable medium, at least one silicone polymer of general formula (I) as defined above, the polymer contained in a sufficient amount in the cosmetic compositions such that the hold of the color towards water of a deposited layer of the cosmetic compositions, once spread over a substrate, is less than or equal to approximately 15.

A fourth embodiment relates to anhydrous cosmetic compositions comprising, in a physiologically acceptable medium, at least one silicone polymer of general formula (I) as defined above, the polymer contained in a sufficient amount in the cosmetic compositions such that the hold of the color towards oil of a deposited layer of the cosmetic compositions, once spread over a substrate, is less than or equal to approximately 25.

A fifth embodiment relates to anhydrous cosmetic compositions comprising, in a physiologically acceptable medium, at least one silicone polymer of general formula (I) as defined above, the polymer contained in a sufficient amount in the cosmetic compositions such that the mean gloss of a deposited layer of the cosmetic compositions, once spread over a substrate, is equal to or greater than approximately 30.

A sixth embodiment relates to anhydrous cosmetic compositions in the cast form comprising, in a physiologically acceptable medium, at least one silicone polymer of general formula (I) as defined above.

A seventh embodiment relates to anhydrous cosmetic compositions comprising, in a physiologically acceptable medium, at least one silicone polymer of general formula (I) as defined above, with the proviso that, when R² is a group of general formula (IIIA):
—C₃H₆—O[CH₂CH(OH)CH₂O]ₙH (IIIA)
in which n is an integer varying from 1 to 5, then R¹ is other than a C₁₋₂ alkyl radical.

Various embodiments relate to cosmetic compositions that are suitable for making up and/or caring for lips and/or skin, for example, lipstick.

Various embodiments relate to methods for making up and/or caring for keratinous substances, such as skin and/or lips, comprising applying at least one cosmetic composition to the keratinous substances.

Various embodiments relate to synthetic substrates on which is present, over all or part of its surface, at least one layer of a cosmetic composition.

Various embodiments relate to preparing anhydrous cosmetic compositions exhibiting an improved hold in combination with a satisfactory or improved mean gloss using at least one silicone polymer of general formula (I) as defined above.

The term “improved hold” is understood to mean an improved hold towards water and/or an improved hold towards oil and/or a reduced transfer and/or a reduced migration.

Various embodiments relate to preparing anhydrous cosmetic compositions exhibiting an improved hold in combination with a satisfactory or improved mean gloss using at least one silicone polymer of general formula (I) as defined above in combination with at least one silicone oil.

In embodiments, the cosmetic compositions can be provided in paste, liquid, gel, cream, or solid form. In embodiments, the cosmetic compositions are in the cast form, for example, in the form of a stick. In embodiments, the disclosed cosmetic compositions can be in the form of a simple or multiple anhydrous emulsion or of a solid or soft anhydrous gel.

The term “composition in the cast form” is understood to mean a solid or semisolid composition obtained on conclusion of the cooling of a composition introduced in the molten state into a mold. In embodiments, the cosmetic composition is in cast the cast form, that is, in a solid or smei-solid form. In embodiments, the disclosed cosmetic compositions can be cast in the form of a stick or into a dish.

A hardness of cast cosmetic compositions can be measured according to the following method. In embodiments, a stick of a cosmetic composition having a circular cross section with a diameter of, for example, approximately 12.7 mm is prepared. The stick is cast and then stored at a temperature of 20° C. for 24 hours before carrying out the measurement.

The hardness can be measured by a “cheese wire” method, in which the stick is transversely cut using a stiff tungsten wire with a diameter of, for example, 250 μm. In embodiments, the wire moves forward at a rate of, for example, 100 mm/min relative to the stick. The hardness corresponds to the maximum shear force exerted by the wire on the stick at 20° C. This force can be measured using, for example, a DFGS2 force gauge sold by Indelco-Chatillon. The hardness can be expressed in grams.

In embodiments, the hardness of the cosmetic compositions presented in the form of a stick can be from approximately 50 to approximately 300 g. In embodiments, the hardness of the cosmetic compositions presented in the form of a stick can be from approximately 70 to approximately 250 g. In embodiments, the cosmetic compositions presented in the form of a stick can be from approximately 100 to approximately 230 g.

In embodiments, the cosmetic compositions exhibit an improved hold of the color, which is reflected, for example, by one or more of: a reduced migration and/or transfer of the color, an improved hold of the color towards water, an improved hold of the color towards oil, and a reduced migration during tracing of the make-up.

In embodiments, the cosmetic compositions maintain a comfortable feel, in the absence of a tacky feel, while exhibiting good adhesion to skin and good flexibility.

In embodiments, the cosmetic compositions maintain an aesthetic effect of the cosmetic compositions, such as the gloss effect, over time.

In embodiments, the cosmetic compositions make it possible to confer a smooth and soft feeling and to maintain a good moisturizing property.

In embodiments, the cosmetic compositions exhibit a good hold in the face of external factors capable of modifying the aesthetic properties thereof, such as sweat, or in the case of a lipstick, a meal.

Silicone Polymer of General Formula (I)

Embodiments of the silicone polymer of general formula (I), which can be used in embodiments of the cosmetic compositions, are disclosed in European Patent Application No. EP 1 213 316.

Silicone polymers of general formula (I) can be used as surface-active agents and/or as oily bases. However, in embodiments, the silicone polymers of general formula (I) suitable for the processing of embodiments of the cosmetic compositions have no role as surface treatment agent.

In embodiments, silicone polymers of general formula (I) improve the hold, the gloss, and/or the comfort of cosmetic compositions.

In embodiments, the silicone polymers used in embodiments of the disclosed cosmetic compositions are represented by the following general formula (I):
R¹ₐR²_{b}R³_{c}SiO_{(4-a-b-c)/2} (I)
in which:
a) a, b and c are such that a varies from 1 to 2.5 and b and c, independently of one another, vary from 0.001 to 1.5,
b) R¹, identical or different, is chosen from:
   C₁ to C₃₀ alkyl radicals, optionally substituted by one or more flu [symbol] atoms or amino and/or carboxyl groups,
   aryl, aralkyl radicals,
   radicals of general formula (II):
      —C_{d}H_{2d}—O—(C₂H₄O)ₑ(C₃H₆O)_{f}R⁴ (II)
   in which:
      R⁴ is a C₁ to C₃₀ hydrocarbon radical or an R⁵—(CO)— radical with R⁵ being a C₁ to C₃₀ hydrocarbon radical, and
      d, e and f are integers such that d varies from 0 to 15 and e and f, independently of one another, vary from 0 to 50, and
   combinations thereof,
c) R² is represented by the following general formula (III):
   -Q—O—X (III)
in which:
   Q is a divalent C₂ to C₂₀ hydrocarbon radical which can include at least one ether bond and/or at least one ester bond, and
   X is a polyhydroxylated hydrocarbon radical,
d) R³ is an organosiloxane group of general formula (IV): 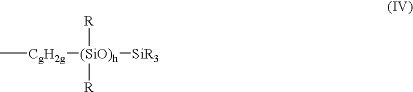
   with:
each of the R radicals representing, independently of one another, a radical chosen from C₁ to C₃₀ alkyl radicals, optionally substituted by one or more fluorine atoms; and aryl and aralkyl radicals,
g and h being integers such that g varies from 1 to 5 and h varies from 0 to 500.

When the R radicals represent a radical chosen from C₁ to C₃₀ alkyl radicals, optionally substituted by one or more fluorine atoms, aryl radicals and aralkyl radicals, they have the same meaning as the R¹ radical as defined above.

In embodiments, the R¹, R² and R³ radicals of the silicone polymers of general formula (I) as defined above are distributed statistically or randomly, that is to say that they do not appear in the structure of the polymer in a specific order. In embodiments, R¹, R² and R³ can respectively represent radicals of different nature in a compound of general formula

In embodiments,
in a):
a can vary from 1.2 to 2.3, and b and c, independently of one another, can vary from 0.05 to 1.

In embodiments,
in b):
R¹ can be an alkyl radical. In embodiments, R¹ can be a C₁ to C₃₀ alkyl radical. In embodiments, R¹ can be a C₁ to C₂₅ alkyl radical. In embodiments, R¹ can be a C₁ to C₂₀ alkyl radical. In embodiments, R¹ can be a C₁ to C₁₀ alkyl radical. In embodiments, R¹ can be a C₁ to C₆ alkyl radical. In embodiments, R¹ can be a C₁ to C₄ alkyl radical. In embodiments, R¹ can be a methyl, ethyl, n-propyl, isopropyl, n-butyl, isobutyl, tert-butyl, pentyl, hexyl, heptyl, octyl, nonyl, decyl, undecyl, or lauryl radical. In embodiments, R¹ can be a cycloalkyl radical, such as a cyclopropyl, a cyclobutyl, a cyclopentyl, or a cyclohexyl. In embodiments, R¹ can be a linear or branched, monounsaturated or polyunsaturated alkyl radical. In embodiments, R¹ can be an alkyl radical substituted by one or more fluorine atoms, such as trifluoropropyl or heptadecafluorodecyl. In embodiments, R¹ can be an alkyl radical substituted by one or more amino groups, such as 2-aminoethyl, 3-aminopropyl, or 3-[(2-aminoethyl)amino]propyl. In embodiments, R¹ can be an alkyl group substituted by one or more carboxyl groups, such as 3-carboxypropyl.
In embodiments, R¹ can be an aryl or aralkyl radical, such as the phenyl radical, the tolyl radical, the benzyl radical and the phenethyl radical.
In embodiments, R¹ can be an organic group represented by the general formula (II):
   —C_{d}H_{2d}—O—(C₂H₄O)ₑ(C₃H₆O)_{f}R⁴ (II)

In embodiments, R¹ can be a hydroxylated radical or a radical resulting from the addition reaction of a linear or branched, saturated or unsaturated, alkenyl ether, in which d=0 and thus of formula:
—O—(C₂H₄O)ₑ(C₃H₆O)_{f}R⁴

In embodiments, e and f are equal to zero, and R¹ is an alkoxy group having from 4 to 30 carbon atoms, for example a lower C₄ to C₁₀ alkoxy radical, such as butoxy or pentoxy, or a higher C₁₁ to C₃₀ alkoxy radical, such as oleoxy or stearoxy, for example, cetyl alcohol, oleyl alcohol and stearyl alcohol, or a radical resulting from an acid or from a fatty acid, such as acetic acid, lactic acid, butyric acid, oleic acid, stearic acid, and behenic acid.

In embodiments, e and f are greater than 1, and R¹ is a hydroxyl radical originating from the addition reaction of an alkylene oxide.

In embodiments, e and f are equal to zero, and d is equal to 3, 5, or 11. In various embodiments, R¹, depending on the nature of the substituent R⁴, is an allyl ether, pentenyl ether or undecenyl ether radical or an allyl stearyl ether, pentenyl behenyl ether or undecenyl oleyl ether radical.

In embodiments, e or f are other than zero, and an alkoxy radical and an ester radical are present via a polyoxyalkylene group.

In embodiments, d is within the range varying from 3 to 5.

In embodiments, the R¹ radical can be any of the radicals defined above or a combination of two or more of these radicals.

In embodiments, R¹ is an alkyl radical chosen from methyl radical, lauryl radical, and combinations thereof.

In embodiments, when R¹ represents two or more radicals (for example a methyl radical and a lauryl radical), these radicals appear randomly in the structure and with a frequency that is specific to them. In embodiments, at least 50% of the R¹ radicals are methyl radicals. In embodiments, at least 70% of the R¹ radicals are methyl radicals. In embodiments, 100% of the R¹ radicals are methyl radicals.

In embodiments,
in c):
   Q can be a divalent hydrocarbon radical chosen from:
      —(CH₂)₂—, —(CH₂)₃—, —CH₂CH(CH₃)—CH₂—, —(CH₂)₄—, —(CH₂)₅—, —(CH₂)₆—, —(CH₂)₇—(CH₂)₈—, —(CH₂)₉—, —(CH₂)₁₀—, —(CH₂)₁₁—, —(CH₂)₂—CH(CH₂CH₂CH₃)—, —CH₂—CH(CH₂CH₃)—, —(CH₂)₃—O—(CH₂)₂—, —(CH₂)₃—O—(CH₂)₂—O—(CH₂)₂—, —(CH₂)₃—O—CH₂CH(CH₃)—, and —CH₂—CH(CH₃)—COO(CH₂)₂—.
   In embodiments, Q is a divalent radical chosen from —(CH₂)₂— and —(CH₂)₃—.
   X can be a polyhydroxylated hydrocarbon radical comprising at least two hydroxyl residues, for example, a hydrocarbon group chosen from glyceryl derivatives and glycoside derivatives.

In embodiments, the glycerol residues can be compounds having the following formulae, in which Q has the same meaning as in the general formula (III) and s and t are integers within the range varying from 1 to 20. In embodiments, s and t are integers within the range varying from 1 to 15. In embodiments, s and t are integers within the range varying from 1 to 10. In embodiments, s and t are integers within the range varying from 1 to 5.

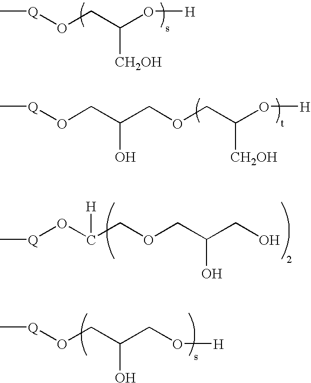

In embodiments, one or more hydroxyl groups of the preceding formulae can be replaced by alkoxy groups or ester groups.

In embodiments, the glycoside radicals which can be used in the general formula (III) can be of monosaccharide type, such as glycosyl, mannosyl, galactosyl, ribosyl, arabinosyl, xylosyl or fructosyl groups. In embodiments, the glycoside radicals which can be used in the general formula (III) can be of oligosaccharide type, such as maltosyl, cellobiosyl, lactosyl or maltotriosyl. In embodiments, the glycoside radicals which can be used in the general formula (II) can be of polysaccharide type, such as cellulose or starch.

In embodiments, the glycoside groups are of monosaccharide or oligosaccharide type.

In embodiments,
in d):
each of the R radicals can represent, independently of one another, a radical chosen from C₁ to C₂₀ alkyl radicals. In embodiments, each of the R radicals can represent, independently of one another, a radical chosen from C₁ to C₁₀ alkyl radicals. In embodiments, each of the R radicals can represent, independently of one another, a radical chosen from C₁ to C₆ alkyl radicals. In embodiments, each of the R radicals can be substituted with one or more fluorine atoms. In embodiments the R radicals represent a radical chosen from the alkyl radicals as defined above, optionally substituted by one or more fluorine atoms, and the R radicals have the same meaning as the R¹ radical as defined above.
in embodiments, g is equal to 2,
in embodiments, h is within the range varying from 1 to 50.

In embodiments, the silicone polymer of general formula (I) is such that the R² radical is represented by the general formula (IIIA):
—C₃H₆O[CH₂CH(OH)CH₂O]ₙH (IIIA)
in which n varies from 1 to 5, and the R¹ radical is other than a C₁₂ alkyl radical.

In embodiments, the silicone polymer of general formula (I) is such that:
a varies from 1 to 1.4, and b and c, independently of one another, vary from 0.02 to 0.04, and
R¹ is a C₁ to C₁₀ alkyl radical, in particular a C₁ to C₆ alkyl radical and more particular a C₁ to C₄ alkyl radical,
R² is represented by the formula (IIIA):
   C₃H₆O[CH₂CH(OH)CH₂O]ₙH (IIIA)
   in which:
n varies from 1 to 5, and
R³ is represented by the formula (IVA):
   —C₂H₄(CH₃)₂SiO[(CH₃)₂SiO]ₘSi(CH₃)₃ (IVA)
   in which:
m varies from 3 to 9.

In embodiments, the silicone polymer of general formula (I), which can be used in embodiments of the cosmetic compositions, is such that:
a varies from 1 to 1.4, and b and c, independently of one another, vary from 0.02 to 0.04,
R¹ is a methyl radical,
R² is represented by the formula (IIIA) in which n varies from 1 to 5, and
R³ is represented by the formula (IVA) in which m varies from 3 to 9.

In embodiments, the silicone polymer of general formula (I) can be chosen from polyglyceryl-3 polydimethylsiloxyethyl dimethicone, lauryl polyglyceryl-3 polydimethylsiloxyethyl dimethicone, and polyglyceryl-3 disiloxane dimethicone, the formulae of which are respectively:
polyglyceryl-3 polydimethylsiloxyethyl dimethicone (formula (V)): 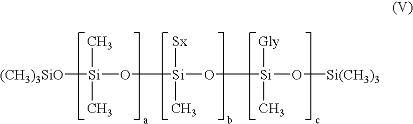
   in which:
Sx: —C₂H₄ [(CH₃)₂SiO]ₘSi(CH₃)₃
Gly: —C₃H₆O[CH₂—CH(OH)CH₂O]ₙH
   with a=1-1.4, b=0.02-0.04, c=0.02-0.04, m=3-9 and n=1-5,
lauryl polyglyceryl-3 polydimethylsiloxyethyl dimethicone (formula (VI)): 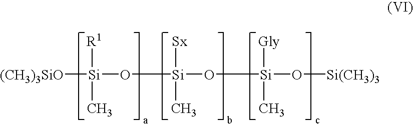
   in which Sx, Gly, a, b, c, m and n have the same meanings as above and R¹ is either a methyl radical or a lauryl radical,
polyglyceryl-3 disiloxane dimethicone (formula (VII)): 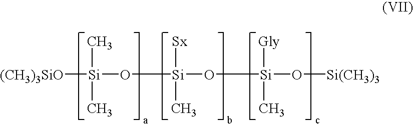
   in which Gly, a, b, c, m and n have the same meanings as above and
Sx: —O(CH₃)₂SiO—Si(CH₃)₃

In embodiments, the silicone polymer of general formula (I) can be present in embodiments of the cosmetic compositions in a proportion of 0.1 to 40% by weight with respect to the total weight of the composition. In embodiments, the silicone polymer of general formula (I) can be present in embodiments of the cosmetic compositions in a proportion of 0.5 to 30% by weight with respect to the total weight of the composition. In embodiments, the silicone polymer of general formula (I) can be present in embodiments of the disclosed cosmetic compositions in a proportion of 1 to 25% by weight with respect to the total weight of the composition. In embodiments, the silicone polymer of general formula (I) can be present in embodiments of the disclosed cosmetic compositions in a proportion of 5 to 20% by weight with respect to the total weight of the composition. In embodiments, the silicone polymer of general formula (I) can be present in embodiments of the disclosed cosmetic compositions in a proportion of 7 to 15% by weight with respect to the total weight of the composition.

In embodiments, the silicone polymer of general formula (I) is employed in a free form. The term “free form” is understood to denote a form of the silicone polymer of general formula (I) in which the polymer is not employed in a form combined with or adsorbed on another material. EP 1 416 016 and EP 1 424 373 disclose examples of silicone polymers that are not in “free form,” and instead are present in the form of a coating of a powder or of a coloring agent in order to block the surface activity of the particles constituting the corresponding powder.

In embodiments, the silicone polymer of general formula (I) is chosen from the polymers sold by Shin-Etsu under the references KF6100®, KF6104® and KF6105®.

Physiologically Acceptable Medium

The term “physiologically acceptable medium” denotes a non-toxic medium suitable for application to skin, lips, or keratinous substances of human beings. The physiologically acceptable medium is also suitable for the substrate to which the composition is applied, and is suitable for the appearance under which the composition is intended to be packaged.

In embodiments, the anhydrous cosmetic compositions can be present in the form of an anhydrous emulsion in which the silicone polymer of general formula (I) as defined above can act as a surfactant.

The term “emulsion” encompasses a lipophilic phase and a hydrophilic phase, the latter not being water.

In embodiments, the cosmetic compositions can be in simple or multiple anhydrous form.

The term “anhydrous cosmetic composition” is understood to mean a composition which can comprise less than 5%, for example less than 3%, less than 2%, and less than 1% of water, with respect to the total weight of the composition. Anhydrous cosmetic compositions can be provided in the form of oily gels, of oily liquids, of pastes, of sticks, and of a vesicular dispersion comprising ionic and/or non-ionic liquids.

Fatty Phase

The term “oil” is understood to mean any fatty substance in the liquid form at ambient temperature (20-25° C.) and at atmospheric pressure.

In embodiments, the cosmetic compositions comprise a fatty phase comprising oils and fatty substances that are solid at ambient temperature (20-25° C.) and atmospheric pressure. In embodiments, the liquid fatty phase comprise, in addition to oils, other compounds dissolved in the oils, such as gelling and/or structuring agents.

In embodiments, the cosmetic compositions can comprise at least one oil. In embodiments, the cosmetic compositions can comprise at least two oils. In embodiments, the oil or oils can be present in the cosmetic compositions in a proportion of 0.1 to 99% by weight with respect to the total weight of the cosmetic compositions. In embodiments, the oil or oils can be present in the cosmetic compositions in a proportion of at least 1 to 90% by weight with respect to the total weight of the cosmetic compositions. In embodiments, the oil or oils can be present in the cosmetic compositions in a proportion of 5 to 70% by weight with respect to the total weight of the cosmetic compositions. In embodiments, the oil or oils can be present in the cosmetic compositions in a proportion of 10 to 60% by weight with respect to the total weight of the cosmetic compositions. In embodiments, the oil or oils can be present in the cosmetic compositions in a proportion of 20 to 50% by weight with respect to the total weight of the cosmetic compositions.

In embodiments, the oil suitable for the preparation of the cosmetic compositions can be volatile or non-volatile, silicone, or non-silicone oils.

The term “volatile oil” is understood to mean an oil (or non-aqueous medium) capable of evaporating on contact with skin in less than one hour at ambient temperature and at atmospheric pressure. The volatile oil is a volatile cosmetic oil that is liquid at ambient temperature and that has a non-zero vapour pressure at ambient temperature and atmospheric pressure, for exmple, a vapour pressure ranging from 0.13 Pa to 40 000 Pa (10-3 to 300 mmHg), ranging from 1.3 Pa to 13 000 Pa (0.01 to 100 mmHg), or ranging from 1.3 Pa to 1300 Pa (0.01 to 10 mmHg).

The term “non-volatile oil” is understood to mean an oil having a vapour pressure of less than 0.13 Pa.

In embodiments, the volatile or non-volatile oils can be hydrocarbon oils, such as oils of animal or vegetable origin, synthetic oils, silicone oils, fluorinated oils, or mixtures thereof.

The term “silicone oil” is understood to mean an oil comprising at least one silicon atom and, for example, at least one Si—O group.

The term “hydrocarbon oil” is understood to mean an oil comprising mainly hydrogen and carbon atoms and optionally oxygen, nitrogen, sulphur, and/or phosphorus atoms.

In embodiments, the volatile hydrocarbon oils can be chosen from hydrocarbon oils having from 8 to 16 carbon atoms. In embodiments, the volatile hydrocarbon oils can be chosen from branched C₈-C₁₆ alkanes (also referred to as isoparaffins), such as isododecane (also referred to as 2,2,4,4,6-pentamethylheptane), isodecane, isohexadecane, and oils sold under the trade names of Isopars® or Permethyls ®.

In embodiments, the volatile silicone oils may be volatile linear or cyclic silicone oils. In embodiments, the volatile linear or cyclic silicone oils may have a viscosity ≧8 centistokes (cSt) (8×10⁻⁶ m²/s). In embodiments, the volatile linear or cyclic silicone oils may have from 2 to 10 silicon atoms. In embodiments, the volatile linear or cyclic silicone oils may have from 2 to 7 silicon atoms. In embodiments, these silicones optionally comprise alkyl or alkoxy groups having from 1 to 10 carbon atoms. In embodiments, the volatile silicone oil may be dimethicones with viscosities of 5 or 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, and mixtures thereof.

In embodiments, the volatile oils may be volatile fluorinated oils, such as nonafluoromethoxybutane or perfluoromethylcyclopentane, and mixtures thereof.

In embodiments, the cosmetic compositions comprise less than 30% by weight of volatile oil with respect to the total weight of the cosmetic composition. In embodiments, the cosmetic compositions comprise less than 15% by weight of volatile oil with respect to the total weight of the cosmetic composition. In embodiments, the cosmetic compositions comprise less than 10% by weight of volatile oil with respect to the total weight of the composition. In embodiments, the cosmetic compositions comprise less than 5% by weight of volatile oil with respect to the total weight of the cosmetic composition.

In embodiments, the cosmetic compositions are devoid of volatile oils.

In embodiments, the fatty phase of the cosmetic compositions comprises at least one non-volatile oil. In embodiments, the non-volatile oils can be chosen from non-volatile hydrocarbon oils, optionally fluorinated, and/or non-volatile silicone oils.

In embodiments, the non-volatile hydrocarbon oil may be:
hydrocarbon oils of animal origin;
hydrocarbon oils of vegetable origin, such as phytosteryl esters (including phytosteryl oleate, phytosteryl isostearate and lauroyl/octyldodecyl/phytosteryl glutamate (Ajinomoto, Eldew PS203)), triglycerides composed of esters of fatty acids and of glycerol, the fatty acids of which can have varied chain lengths from C₄ to C₂₄ (it being possible for these chains to be linear or branched and saturated or unsaturated) (including heptanoic or octanoic triglycerides), wheat germ, sunflower, grape seed, sesame, maize, apricot, castor, shea, avocado, olive, soybean, sweet almond, palm, rapeseed, cottonseed, hazelnut, macadamia, jojoba, alfalfa, poppy, pumpkinseed, cucumber, blackcurrant seed, evening primrose, millet, barley, quinoa, rye, safflower, Kendal nut, passionflower or musk rose oil, shea butter, and triglycerides of caprylic/capric acids, such as those sold by Stearineries Dubois or those sold under the names Miglyol 810®, 812® and 818® by Dynamit Nobel;
synthetic ethers having from 10 to 40 carbon atoms;
linear or branched hydrocarbons of mineral or synthetic origin, such as liquid petrolatum, polydecenes, hydrogenated polyisobutene, such as parleam, squalane, and mixtures thereof;
synthetic esters, such as oils of formula R₁COOR₂ in which R₁ represents the residue of a linear or branched fatty acid comprising from 1 to 40 carbon atoms, and R₂ represents a hydrocarbon chain, such as a branched hydrocarbon chain, comprising from 1 to 40 carbon atoms, provided that R₁+R₂≧10.

In embodiments, the esters can be chosen from fatty acid esters, such as:
cetearyl octanoate, isopropyl alcohol esters, such as isopropyl myristate, isopropyl palmitate, ethyl palmitate, 2-ethylhexyl palmitate, isopropyl stearate or isostearate, isostearyl isostearate, octyl stearate, hydroxylated esters, such as isostearyl lactate, octyl hydroxystearate, diisopropyl adipate, heptanoates and in particular isostearyl heptanoate, octanoates, decanoates or ricinoleates of alcohols or of polyalcohols, such as propylene glycol dioctanoate, cetyl octanoate, tridecyl octanoate, 2-ethylhexyl palmitate and 4-diheptanoate, alkyl benzoate, polyethylene glycol diheptanoate, propylene glycol di(2-ethylhexanoate) and mixtures thereof, benzoates of C₁₂ to C₁₅ alcohols, hexyl laurate, neopentanoic acid esters, such as isodecyl neopentanoate, isotridecyl neopentanoate, isostearyl neopentanoate or octyldodecyl neopentanoate, esters of isononanoic acid, such as isononyl isononanoate, isotridecyl isononanoate or octyl isononanoate, or hydroxylated esters, such as isostearyl lactate or diisostearyl malate;
esters of polyols and esters of pentaerythritol, such as dipentaerythritol tetrahydroxystearate/tetraisostearate;
esters of dimer diols and dimer diacids, such as Lusplan DD-DA5® and Lusplan DD-DA7®, sold by Nippon Fine Chemical and disclosed in French Patent Application No. FR0302809, filed on 6 Mar. 2003;
fatty alcohols which are liquid at ambient temperature with a branched and/or unsaturated carbon chain having from 12 to 26 carbon atoms, such as 2-octyldodecanol, isostearyl alcohol, oleyl alcohol, 2-hexyldecanol, 2-butyloctanol and 2-undecylpentadecanol;
higher fatty acids, such as oleic acid, linoleic acid, linolenic acid and mixtures thereof; and
dialkyl carbonates, with two different or identical alkyl chains, such as dicaprylyl carbonate, sold under the name Cetiol CC® by Cognis.

In embodiments, the non-volatile silicone oils can be non-volatile polydimethylsiloxanes (PDMSs), polydimethylsiloxanes comprising pendant alkyl or alkoxy groups and/or alkyl or alkoxy groups at the ends of the silicone chain (each group having from 2 to 24 carbon atoms), phenylated silicones, such as phenyl trimethicones, phenyl dimethicones, phenyl(trimethylsiloxy)diphenylsiloxanes, diphenyl dimethicones, diphenyl(methyldiphenyl)trisiloxanes and (2-phenylethyl)trimethylsiloxysilicates, dimethicones or phenyl trimethicones with a viscosity of less than or equal to 100 cSt, and mixtures thereof.

In embodiments, the cosmetic compositions comprise at least one silicone oil chosen from volatile silicone oils, non-volatile silicone oils, and mixtures thereof.

In embodiments, the non-volatile oil is chosen from hydrogenated polyisobutene, isostearyl heptanoate, isononyl isononanoate, isotridecyl isononanoate, diisostearyl malate, dipentaerythritol tetrahydroxystearate/tetraisostearate, 2-octyldodecanol, and mixtures thereof.

In embodiments, the non-volatile oil present in the cosmetic composition is a mixture of hydrogenated polyisobutene, of isostearyl heptanoate, of isononyl isononanoate, of isotridecyl isononanoate, of diisostearyl malate, of dipentaerythritol tetrahydroxystearate/tetraisostearate, and of 2-octyldodecanol.

In embodiments, the non-volatile oils can be present in the cosmetic compositions in a content ranging from 20% to 99% by weight with respect to the total weight of the cosmetic compositions. In embodiments, the non-volatile oils can be present in the cosmetic compositions in a content ranging from 30% to 80% by weight with respect to the total weight of the cosmetic compositions. In embodiments, the non-volatile oils can be present in the cosmetic compositions in a content ranging from 40% to 80% by weight with respect to the total weight of the cosmetic composition.

In embodiments, the liquid fatty phase of the cosmetic compositions is a silicone oil present in the cosmetic compositions in a content ranging from 0 to 90% by weight with respect to the total weight of the cosmetic compositions. In embodiments, the silicone oil is present in the cosmetic compositions in a content ranging from 0.1 to 80% by weight with respect to the total weight of the cosmetic compositions. In embodiments, the silicone oil is present in the cosmetic compositions in a content ranging from 2 to 80% by weight with respect to the total weight of the cosmetic composition.

In embodiments, the silicone oil is present in the cosmetic compositions at a ratio by weight, with respect to the silicone polymer of general formula (I), varying from 80:1. In embodiments, the silicone oil is present in the cosmetic compositions at a ratio by weight, with respect to the silicone polymer of general formula (I), varying from 60:1. In embodiments, the silicone oil is present in the cosmetic compositions at a ratio by weight, with respect to the silicone polymer of general formula (I), varying from 40:1.

In embodiments, the cosmetic compositions comprise a non-volatile fluid silicone compound.

In embodiments, the liquid fatty phase can be thickened, gelled, or structured by incorporating therein a fatty-phase gelling agent, as disclosed in International Patent Application No. WO 2004/55080, published on 1 Jul. 2004.

In embodiments, the cosmetic compositions comprise at least one compound chosen from waxes, pasty fatty substances, and mixtures thereof.

In embodiments, the wax is solid at ambient temperature (25° C.), with a reversible solid/liquid change of state. In embodiments, the wax has a melting point of greater than 30° C., ranging up to 200° C. In embodiments, the wax has a hardness of 0.5 MPa and exhibits, in the solid state, an anisotropic crystalline arrangement. In embodiments, the wax can be a hydrocarbon, fluorinated, and/or a silicone wax. In embodiments, the wax can be of animal, vegetable, mineral, or synthetic origin. In embodiments, the wax can be chosen from beeswax, carnauba wax, candelilla wax, paraffin waxes, hydrogenated castor oil, synthetic waxes, including polyethylene waxes (such as polyethylene waxes having a molecular weight of between 400 and 600) or Fischer-Tropsch waxes, silicone waxes, such as alkyl or alkoxy dimethicones having from 16 to 45 carbon atoms, ceresines or ozokerites, including isoparaffins having a melting point of less than 40° C., such as EMW-0003, sold by Nippon Seirou, α-olefin oligomers, such as the polymers Performa V® 825, 103 and 260, sold by New Phase Technologies, ethylene-propylene copolymers, such as Performalene®EP 700, microcrystalline waxes having a melting point of greater than 85° C., such as Hi-Mic® 1070, 1080, 1090 and 3080, sold by Nippon Seirou, and mixtures thereof.

In embodiments, the wax is chosen from polyethylene waxes, candelilla wax, and mixtures thereof. In embodiments, the cosmetic compositions comprise a mixture of polyethylene wax and candelilla wax.

In embodiments, the wax or waxes is or are present in the cosmetic compositions in a content varying from approximately 1.5 to approximately 20% by weight with respect to the total weight of the cosmetic compositions. In embodiments, the wax or waxes is or are present in the cosmetic compositions in a content varying from approximately 3 to approximately 15% by weight with respect to the total weight of the cosmetic compositions. In embodiments, the wax or waxes is or are present in the cosmetic compositions in a content varying from approximately 5 to approximately 10% by weight with respect to the total weight of the cosmetic compositions. In embodiments, the wax or waxes is or are present in the cosmetic compositions in a content varying from approximately 6.5 to approximately 8.5% by weight with respect to the total weight of the cosmetic compositions.

The term “pasty” is understood to mean a fatty compound with a reversible solid/liquid change of state comprising, at a temperature of 23° C., a liquid fraction and a solid fraction. For example, poly(vinyl laurate) is “pasty.” In embodiments, the cosmetic compounds comprise at least one pasty compound.

In embodiments, the pasty compounds exhibit a hardness at 20° C. ranging from approximately 0.001 to approximately 0.5 Mpa. In embodiments, the pasty compounds exhibit a hardness at 20° C. ranging from approximately 0.002 to approximately 0.4 MPa.

In embodiments, the pasty compounds may be lanolins and lanolin derivatives, such as acetylated lanolins, oxypropylenated lanolins or isopropyl lanolate, and mixtures thereof. In embodiments, the pasty compounds may be fatty acids esters or fatty alcohols esters, including those having 20 to 65 carbon atoms, such as triisostearyl or cetyl citrate, arachidyl propionate, poly(vinyl laurate, cholesterol esters, such as triglycerides of vegetable origin, (including hydrogenated vegetable oils), viscous polyesters, and mixtures thereof. In embodiments, the pasty compound may be a triglyceride of vegetable origin, for example hydrogenated castor oil derivatives, such as “Thixin®” from Rheox.

In embodiments, the pasty compounds may be polyesters resulting from the esterification of a carboxylic acid and of an aliphatic hydroxycarboxylic acid ester, for example Risocast® DA-L (an ester resulting from the esterification reaction of hydrogenated castor oil with dilinoleic acid in proportions of 2 to 1) and Risocast® DA-H (an ester resulting from the esterification of hydrogenated castor oil with isostearic acid in proportions of 4 to 3), sold by the Japanese company Kokyu Alcohol Kogyo.

In embodiments, the pasty compounds may be hydrogenated coco-glycerides.

In embodiments, the pasty compounds may be pasty silicone compounds, such as polydimethylsiloxanes (PDMSs). In embodiments, the pasty silicone compounds may have a high molecular weights, may have pendant chains of the alkyl or alkoxy type having from 8 to 24 carbon atoms, and may have a melting point of 20-55° C., such as stearyl dimethicones, for example those sold by Dow Corning under the trade names DC2503® and DC25514®, and mixtures thereof.

Hydrophilic Phase

In embodiments, the cosmetic compositions comprise at least one hydrophilic phase that can form the continuous phase of the anhydrous composition. In embodiments, the hydrophilic phase is essentially devoid of water.

In embodiments, the hydrophilic phase comprises a mixture of water-miscible organic solvents (miscibility in water of greater than 50% by weight at 25° C.), such as lower monoalcohols having from 1 to 5 carbon atoms, for example ethanol or isopropanol; glycols having from 2 to 8 carbon atoms, for example propylene glycol, ethylene glycol, 1,3-butylene glycol or dipropylene glycol; C₃-C₄ ketones; and C₂-C₄ aldehydes.

In embodiments, the hydrophilic phase can be present at a content ranging from 0.1 to 40% by weight with respect to the total weight of the cosmetic composition. In embodiments, the hydrophilic phase can be present at a content ranging from 0.1 to 20% by weight with respect to the total weight of the cosmetic composition. In embodiments, the hydrophilic phase can be present at a content ranging from 0.1 to 10% by weight with respect to the total weight of the cosmetic composition.

Polyols

In embodiments, the cosmetic compositions comprise at least one polyol or polyhydric alcohol.

The term “polyhydric alcohol” or “polyol” should be understood as meaning any organic molecule comprising at least two free hydroxyl groups.

In embodiments, the cosmetic compositions contain polyhydric alcohols that have from 2 to 20 carbon atoms. In embodiments, the cosmetic compositions contain polyhydric alcohols that have from 2 to 10 carbon atoms. In embodiments, the cosmetic compositions contain polyhydric alcohols that have from 2 to 6 carbon atoms.

In embodiments, the cosmetic compositions contain polyols chosen from glycerol, propylene glycol, butylene glycol, pentylene glycol, hexylene glycol, dipropylene glycol, diethylene glycol, sorbitol, hydroxypropyl sorbitol, 1,2,6-hexanetriol, glycol ethers (having in particular from 3 to 16 carbon atoms), such as mono-, di- or tripropylene glycol (C₁-C₄)alkyl ethers or mono-, di- or triethylene glycol (C₁-C₄)alkyl ethers, and mixtures thereof.

Film-Forming Agents

In embodiments, the cosmetic compositions comprise at least one film-forming agent, optionally in combination with at least one additional agent that is able to form a film. In embodiments, the film-forming agents comprise film-forming polymers.

The term “film-forming polymer” denotes a polymer capable of forming, alone or in the presence of an additional agent that is able to form a film, an isolable film, including a continuous film that adheres to a substrate (such as to keratinous substances), a cohesive film, and a film with the cohesion and mechanical properties such that the film can be isolated from the substrate.

In embodiments, the cosmetic compositions may contain a single film-forming polymer or a blend of film-forming polymers. In embodiments, the film-forming polymer(s) can be chosen from the group consisting of synthetic polymers of radical polymer or polycondensate type and polymers of natural origin and their blends, such as those disclosed in French Patent Application No. FR0450540, filed on 18 Mar. 2004.

In embodiments, the content of film-forming agents in the cosmetic compositions can vary from approximately 0.1 to approximately 20% by weight with respect to the total weight of the cosmetic compositions. In embodiments, the content of film-forming agents in the cosmetic compositions can vary from approximately 0.5 to approximately 20% by weight with respect to the total weight of the cosmetic compositions. In embodiments, the content of film-forming agents in the cosmetic compositions can vary from approximately 1 to approximately 20% by weight with respect to the total weight of the cosmetic compositions.

In embodiments, the film-forming agent is present in the cosmetic compositions in a ratio by weight, with respect to the weight of the silicone polymers of general formula (I), of less than 5:1. In embodiments, the film-forming agent is present in the cosmetic compositions in a ratio by weight, with respect to the weight of the silicone polymers of general formula (I), of less than 1:1. In embodiments, , the film-forming agent is present in the cosmetic compositions in a ratio by weight, with respect to the weight of the silicone polymers of general formula (I), of less than or equal to 1:2.

In embodiments, the film-forming polymers may be acrylic polymers and copolymers, polyurethanes, polyesters, polyamides, polyureas, cellulose polymers, such as nitrocellulose, and silicone polymers.

In embodiments, the cosmetic compositions may contain silicone resins, which are crosslinked polyorganosiloxane polymers generally soluble or swellable in silicone oils. In embodiments, the silicone resins may be siloxysilicates, polysilsesquioxanes (such as the polymethylsesquioxane resins sold by Wacker under the reference Resin MK, such as Belsil PMS MK, or those sold by Shin-Etsu, for example under the KR-220L references), and polymethylsesquioxanes (such as the trimethylsiloxysilicate resins sold under the reference SR1000 by General Electric or under the reference TMS803 by Wacker, and the trimethylsiloxysilicate resins sold under the name KF-7312J by Shin-Etsu and the DC749 and DC593 resins sold by Dow Corning).

In embodiments, the cosmetic compositions may contain fat-soluble silicone polymers. In embodiments, the fat-soluble silicone polymers can be silicone polyamides of the polyorganosiloxane type, such as those disclosed in the U.S. Pat. Nos. 5,874,069, 5,919,441, 6,051,216, and 5,981,680.

In embodiments, the film-forming polymer can be chosen from polymers having a non-silicone organic backbone grafted with monomers comprising a polysiloxane. These polymers can be fat-soluble, fat-dispersible, or water-soluble polymers, or these polymers can be polymers that are dispersible in an aqueous medium. Furthermore, these polymers are composed of a main organic chain formed of organic monomers not comprising silicone, and at least one polysiloxane macromer is grafted to monomers within the chain, and optionally on at least one of the ends of the chain.

The expression “polysiloxane macromer” denotes any monomer comprising a polymer chain of the polysiloxane type in its structure. In embodiments, the non-silicone organic monomers constituting the main chain of the grafted silicone polymer can be chosen from monomers possessing ethylenic unsaturation which can be polymerized by the radical method, monomers which can be polymerized by polycondensation such as those forming polyamides, polyesters or polyurethanes, or ring-opening monomers, such as those of the oxazoline or caprolactone type.

In embodiments, the polymers having a non-silicone organic backbone grafted with monomers comprising a polysiloxane can be obtained with any method known to a person skilled in the art. In embodiments, the polymers having a non-silicone organic backbone grafted with monomers comprising a polysiloxane can be obtained by the reaction between (i) a starting polysiloxane macromer correctly functionalized on the polysiloxane chain and (ii) one or more non-silicone organic compounds, themselves correctly functionalized by a functional group which is capable of reacting with the functional group or groups carried by the silicone with the formation of a covalent bond, for example, the radical reaction between a vinyl group carried at one of the ends of the silicone and a double bond of a monomer possessing ethylenic unsaturation of the main chain.

In embodiments, the polymers having a non-silicone organic backbone grafted with monomers comprising a polysiloxane are chosen from those disclosed in U.S. Pat. Nos. 4,693,935, 4,728,571, and 4,972,037, European Patent Applications Nos. EP-A-0 412 704, EP-A-0 412 707, EP-A-0 640 105, and International Application No. WO 95/00578. In embodiments, copolymers are obtained by radical polymerization starting from monomers possessing ethylenic unsaturation and from monomers having a vinyl end group. In embodiments, copolymers are obtained by the reaction of a polyolefin comprising functionalized groups and a polysiloxane macromer having an end functional group which reacts with the functionalized groups.

In embodiments, the grafted silicone polymers comprise:
a) from 0 to 98% by weight of at least one lipophilic monomer (A) of low polarity possessing ethylenic unsaturation which can be polymerized by the radical method;
b) from 0 to 98% by weight of at least one hydrophilic polar monomer (B) possessing ethylenic unsaturation which can be copolymerized with the monomer or the monomers of the type (A);
c) from 0.01 to 50% by weight of at least one polysiloxane macromer (C) of general formula (XXVII):
   X(Y)ₙSi(R)₃₋ₘZₘ (XXVII)
   in which:
X denotes a vinyl group which can be copolymerized with the monomers (A) and (B);
Y denotes a group having a divalent bond;
R denotes hydrogen, C₁-C₆ alkyl or alkoxy, C₆-C₁₂ aryl;
Z denotes a monovalent polysiloxane unit having a number-average molecular weight of at least 500;
n has the value 0 or 1 and m is an integer ranging from 1 to 3; the percentages being calculated with respect to the total weight of the monomers (A), (B) and (C).

In embodiments, the grafted silicone polymers have a number-average molecular weight ranging from approximately 10 000 to approximately 2 000 000. In embodiments, the grafted silicone polymers have a glass transition temperature Tg or a crystalline melting point M.p. of at least −20° C.

In embodiments, the lipophilic monomers (A) may be esters of C₁-C₁₈ alcohol and of acrylic or methacrylic acid; esters of C₁₂C₃₀ alcohol and of methacrylic acid; styrene; polystyrene macromers; vinyl acetate; vinyl propionate; α-methylstyrene; tert-butylstyrene; butadiene; cyclohexadiene; ethylene; propylene; vinyltoluene; esters of acrylic or methacrylic acid and of 1,1-dihydroperfluoroalkanols or of homologues of the latter; esters of acrylic or methacrylic acid and of ω-hydrofluoroalkanols; esters of acrylic or methacrylic acid and of fluoroalkylsulphonamido alcohols; esters of acrylic or methacrylic acid and of fluoroalkyl alcohols; esters of acrylic or methacrylic acid and of alcohol fluoroethers; and mixtures of these. In embodiments, the lipophilic monomers (A) are chosen from the group consisting of n-butyl methacrylate, isobutyl methacrylate, tert-butyl acrylate, tert-butyl methacrylate, 2-ethylhexyl methacrylate, methyl methacrylate, 2-(N-methylperfluorooctanesulphonamido)ethyl acrylate, 2-(N-butylperfluorooctanesulphonamido)ethyl acrylate, and mixtures of these.

In embodiments, the polar monomers (B) may be acrylic acid, methacrylic acid, N,N-dimethylacrylamide, dimethylaminoethyl methacrylate, quaternized dimethylaminoethyl methacrylate, (meth)acrylamide, N-(t-butyl)acrylamide, maleic acid, maleic anhydride and the hemiesters of these, hydroxyalkyl (meth)acrylates, diallyldimethylammonium chloride, vinylpyrrolidone, vinyl ethers, maleimides, vinylpyridine, vinylimidazole, vinyl and heterocyclic polar compounds, styrenesulphonate, allyl alcohol, vinyl alcohol, vinylcaprolactam, and mixtures of these. In embodiments, the polar monomers (B) are chosen from the group consisting of acrylic acid, N,N-dimethylacrylamide, dimethylaminoethyl methacrylate, quaternized dimethylaminoethyl methacrylate, vinylpyrrolidone, and mixtures of these.

In embodiments, the grafted silicone polymers are KP 561 or KP 562, sold by Shin-Etsu, such that the monomer (A) is chosen from esters of C₁₈ to C₂₂ alcohol and of methacrylic acid.

Coloring Materials

In embodiments, the cosmetic compositions can incorporate one or more coloring agents. In embodiments, the coloring agents can be at least one organic or inorganic coloring material, such as pigments or pearlescent agents.

The term “pigments” should be understood as meaning white or colored, inorganic or organic particles that are insoluble in an aqueous solution and that are suitable for coloring and/or opacifying the resulting film.

In embodiments, the pigments can be present in the cosmetic compositions in a proportion of 0.01 to 15% by weight with respect to the total weight of the cosmetic composition. In embodiments, the pigments can be present in the cosmetic compositions in a proportion of 0.01 to 10% by weight with respect to the total weight of the cosmetic composition. In embodiments, the cosmetic compositions in a proportion of 0.02 to 5% by weight with respect to the total weight of the cosmetic composition. In embodiments, the pigments may be mineral pigments, such as titanium, zirconium or cerium oxides, zinc, iron or chromium oxides, ferric blue, manganese violet, ultramarine blue, and chromium hydrate.

In embodiments, the pigments can have a structure, for example, of a sericite/brown iron oxide/titanium dioxide/silica type. Examples of such pigments are sold under the reference Coverleaf NS or JS by Chemicals and Catalysts, and exhibit a contrast ratio in the region of approximately 30.

In embodiments, the coloring material comprises a pigment having a structure, for example, of the type of silica microspheres comprising iron oxide. An example of pigment exhibiting this structure is that sold by Miyoshi under the reference PC Ball PC-LL-100 P, this pigment being composed of silica microspheres comprising yellow iron oxide.

In embodiments, the organic pigments can be carbon black, pigments of D & C type, lakes based on cochineal carmine of barium, strontium, calcium or aluminium, or the diketopyrrolopyrroles (DPP) disclosed in the documents EP-A-542669, EP-A-787730, EP-A-787731 and WO-A-96/08537.

The term “pearlescent agents” should be understood as meaning colored particles of any shape, which may or may not be iridescent, for example produced by certain shellfish in their shells or synthesized. “Pearlescent agents” exhibit a coloring effect by optical interference.

In embodiments, the pearlescent agents can be chosen from pearlescent pigments, such as titanium oxide-coated mica covered with an iron oxide, titanium oxide-coated mica covered with bismuth oxychloride, titanium oxide-coated mica covered with chromium oxide, titanium oxide-coated mica covered with an organic dye, and pearlescent pigments based on bismuth oxychloride. In embodiments, the pearlescent agents can be mica particles, at the surface of which at least two successive layers of metal oxides and/or of organic coloring materials are superimposed. In embodiments, the pearlescent agents can be natural mica covered with titanium oxide, with iron oxide, with natural pigment or with bismuth oxychloride.

Examples of suitable pearlescent agents include the Timica, Flamenco and Duochrome (mica-based) pearlescent agents sold by Engelhard, the Timiron pearlescent agents sold by Merck, the Prestige mica-based pearlescent agents sold by Eckart, and the Sunshine synthetic mica-based pearlescent agents sold by Sun Chemical.

In embodiments, the pearlescent agents can have a yellow, pink, red, bronze, orangey, brown, gold, and/or coppery color or glint.

In embodiments, the pearlescent agents can be pearlescent agents of gold color sold by Engelhard under the name of Brillant Gold 212G (Timica), Gold 222C (Cloisonne), Sparkle Gold (Timica), Gold 4504 (Chromalite) and Monarch Gold 233X (Cloisonne); bronze pearlescent agents sold by Merck under the names Bronze Fine (17384) (Colorona) and Bronze (17353) (Colorona) and by Engelhard under the name Super Bronze (Cloisonne); orange pearlescent agents sold by Engelhard under the names Orange 363C (Cloisonne) and Orange MCR 101 (Cosmica) and by Merck under the names Passion Orange (Colorona) and Matte Orange (17449) (Microna); brown-colored pearlescent agents sold by Engelhard under the names Nu Antique Copper 340XB (Cloisonne) and Brown CL4509 (Chromalite); pearlescent agents with a copper glint sold by Engelhard under the name Copper 340A (Timica); pearlescent agents with a red glint sold by Merck under the name Sienna Fine (17386) (Colorona); pearlescent agents with a yellow glint sold by Engelhard under the name Yellow (4502) (Chromalite); red-colored pearlescent agents with a gold glint sold by Engelhard under the name Sunstone G012 (Gemtone); pink pearlescent agents sold by Engelhard under the name Tan Opale G005 (Gemtone); black pearlescent agents with a gold glint sold by Engelhard under the name Nu Antique Bronze 240 AB (Timica); blue pearlescent agents sold by Merck under the name Matte Blue (17433) (Microna); white pearlescent agents with a silvery glint sold by Merck under the name Xirona Silver; gold and green pinkish orangey pearlescent agents sold by Merck under the name Indian Summer (Xirona); and mixtures thereof.

In embodiments, the cosmetic compositions comprise water-soluble or fat-soluble dyes. In embodiments, the cosmetic compositions comprise water-soluble or fat-soluble dyes in a content ranging from 0.01 to 10% by weight with respect to the total weight of the cosmetic composition. In embodiments, the cosmetic compositions comprise water-soluble or fat-soluble dyes in a content ranging from 0.01 to 5% by weight with respect to the total weight of the cosmetic composition. In embodiments, the fat-soluble dyes can be Sudan red, DC Red 17, DC Green 6, β-carotene, soybean oil, Sudan brown, DC Yellow 11, DC Violet 2, DC Orange 5, or quinoline yellow. In embodiments, the water-soluble dyes can be beetroot juice or methylene blue.

In embodiments, the cosmetic compositions comprise at least one material with a specific optical effect. The special optical effect is different from a simple conventional coloring effect, that is to say different from a unified and stabilized effect such as that produced by conventional coloring materials (for example, monochromatic pigments). The term “stabilized” means devoid of an effect of variability in the color with the angle of observation or in response to a change in temperature.

In embodiments, the material with a special optical effect can be chosen from particles with a metallic glint, goniochromatic coloring agents, diffracting pigments, thermochromic agents, optical brighteners, and fibres (such as interferential fibres). In embodiments, these various materials can be combined so as to provide the simultaneous display of two or more effects.

In embodiments, the particles with a metallic glint can be chosen from:
particles of at least one metal and/or of at least one metal derivative,
particles comprising an organic or inorganic substrate, made of one or more materials, at least partially covered with at least one layer with a metallic glint comprising at least one metal and/or at least one metal derivative, and
mixtures of the particles.
   The term “metal derivatives” denotes compounds derived from metals, such as oxides, fluorides, chlorides, and sulphides. In embodiments, the particles can contain metals such as Ag, Au, Cu, Al, Ni, Sn, Mg, Cr, Mo, Ti, Zr, Pt, Va, Rb, W, Zn, Ge, Te, Se, and mixtures or alloys thereof (for example, bronzes and brasses). In embodiments, the particles may be aluminium particles, such as those sold under the names Starbrite 1200 EAC® by Siberline and Metalure® by Eckart.

In embodiments, the particles may contain metal powders formed of copper or alloy mixtures (such as the references 2844 sold by Radium Bronze), metal pigments (such as aluminium or bronze, for example those sold under the names Rotosafe 700 from Eckhart), silica-coated aluminium particles (such as those sold under the name Visionaire Bright Silver from Eckhart), and particles formed of metal alloy (such as powders formed of bronze (copper and zinc alloy) coated with silica, for example those sold under the name Visionaire Bright Natural Gold from Eckart).

In embodiments, the particles comprise a glass substrate, such as those sold by Nippon Sheet Glass under the names Microglass Metashine.

In embodiments, the goniochromatic coloring agent can be chosen from interferential multilayered structures and liquid crystal coloring agents.

In embodiments, the cosmetic compositions may contain symmetrical interferential multilayer structures, such as: Al/SiO₂/Al/SiO₂/Al, pigments having this structure being sold by DuPont de Nemours; Cr/MgF₂/Al/MgF₂/Cr, pigments having this structure being sold under the name Chromaflair by Flex; MoS₂/SiO₂/Al/SiO₂/MoS₂; Fe₂O₃/SiO₂ μl/SiO₂/Fe₂O₃ and Fe₂O₃/SiO₂/Fe₂O₃/SiO₂/Fe₂O₃, pigments having these structures being sold under the name Sicopearl by BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name Xirona by Merck (Darmstadt). In embodiments, the pigments can be pigments with a silica/titanium oxide/tin oxide structure sold under the name Xirona Magic by Merck, pigments with a silica/brown iron oxide structure sold under the name Xirona Indian Summer by Merck and pigments with a silica/titanium oxide/mica/tin oxide structure sold under the name Xirona Caribbean Blue by Merck. In embodiments, the pigments can be Infinite Colors pigments from Shiseido. D ifferent effects are obtained according to the thickness and the nature of the various layers. Thus, with the structure Fe₂O₃/SiO₂/Al/SiO₂/Fe₂O₃, the color changes from green-golden to red-grey for SiO₂ layers of 320 to 350 nm; from red to golden for SiO₂ layers of 380 to 400 nm; from purple to green for SiO₂ layers of 410 to 420 nm; and from copper to red for SiO₂ layers of 430 to 440 nm.

In embodiments, of the cosmetic compositions may contain pigments with a polymeric multilayer structure, such as those sold by 3M under the name Color Glitter. In embodiments, of the cosmetic compositions may contain liquid crystal goniochromatic particles, such as those sold by Chenix and of those sold under the name Helicone® HC by Wacker.

Fillers

In embodiments, the cosmetic compositions comprise at least one filler. In embodiments, the filler is of an organic or mineral nature, which confers thereon an improved stability with regard to exudation.

The term “filler” should be understood as meaning colorless or white solid particles of any shape that are provided in an insoluble form dispersed in the medium of embodiments of the cosmetic compositions. Fillers of a mineral or organic nature confer body or stiffness on the cosmetic compositions and/or softness, mattness and uniformity on the make-up.

In embodiments, the fillers can have lamellar, globular or spherical shape, or be in the form of fibres or other forms intermediate between these defined forms.

In embodiments, the fillers may or may not be surface-coated. In embodiments, the fillers can be surface-treated with silicones, amino acids, fluorinated derivatives, or other substances that promote the dispersion and the compatibility of the filler in the cosmetic compositions.

In this disclosure, the terms “mineral fillers” and “inorganic fillers” are user interchangeably.

In embodiments, the mineral fillers can be talc, mica, silica, trimethylsiloxysilicate, kaolin, bentone, precipitated calcium carbonate, magnesium carbonate and hydrogen carbonate, hydroxyapatite, boron nitride, hollow silica microspheres (Silica Beads from Maprecos), glass or ceramic microcapsules, silica-based fillers (such as Aerosil 200 or Aerosil 30,; Sunsphare L-31 or Sunphare H-31 (sold by Asahi Glass), Chemicelen (sold by Asahi Chemical), composites of silica and of titanium dioxide (such as the TSG series sold by Nippon Sheet Glass), and mixtures thereof.

In embodiments, the organic fillers can be polyamide powders (Nylon® Orgasol from Atochem), poly-β-alanine powders, polyethylene powders, polytetrafluoroethylene (Teflon®) powders, lauroyllysine, starch, powders formed of tetrafluoroethylene polymers, hollow microspheres formed of polymers, such as Expancel (Nobel Industrie), precipitated calcium carbonate, magnesium carbonate and hydrogen carbonate, metal soaps derived from organic carboxylic acids having from 8 to 22 carbon atoms, metal soaps derived from organic carboxylic acids having from 12 to 18 carbon atoms, for example zinc stearate, magnesium stearate, lithium stearate, zinc laurate or magnesium myristate, Polypore® L 200 (Chemdal Corporation), silicone resin microbeads (Tospearl® from Toshiba, for example) or polyurethane powders, such as powders formed of crosslinked polyurethane comprising the copolymer trimethylol hexyllactone, and mixtures thereof. In embodiments, the organic fillers can be hexamethylene diisocyanate/trimethylol hexyllactone polymers, such as those sold under the name of Plastic Powder D-400® or Plastic Powder D-800® from Toshiki.

In embodiments, the fillers can be present in the cosmetic compositions in a proportion of 0.001 to 35% of the total weight of the cosmetic compositions. In embodiments, the fillers can be present in the cosmetic compositions in a proportion of 0.5 to 15% of the total weight of the cosmetic compositions.

In embodiments, the filler can have a mean particle size of less than approximately 100 μm. In embodiments, the filler can have a mean particle size of between approximately 1 and approximately 50 μm. In embodiments, the filler can have a mean particle size of between approximately 4 and approximately 20 μm.

In embodiments, the cosmetic compositions comprise at least one filler present in a proportion of 0.01% to 60% of the total weight of the cosmetic compositions. In embodiments, the cosmetic compositions comprise at least one filler present in a proportion of 0.5 to 20% of the total weight of the cosmetic compositions. In embodiments, the cosmetic compositions comprise at least one filler present in a proportion of 1 to 10%, by weight of the total weight of the composition.

Additives

In embodiments, the cosmetic compositions comprise additives conventionally used in the field concerned. In embodiments, the cosmetic compositions comprise additives chosen from gelling agents, such as disclosed in International Patent Application No. WO 2004/55080, surface-active agents, such as disclosed in French Patent Application No. FR 2 834 452, gums, antioxidants, essential oils, preservatives, fragrances, neutralizing agents, moisturizing agents, antiseptics, vitamins, such as vitamin B3 or vitamin E and their derivatives, and agents which protect against UV radiation.

In embodiments, the additives comprise silicone surfactants that are of non-crosslinked type. In embodiments, the cosmetic compositions are devoid of surfactants belonging to the family of ammonium type surfactants.

In embodiments, the silicone polymer of general formula (I) is chosen from polyglyceryl-3 polydimethylsiloxyethyl dimethicone, lauryl polyglycerol-3 polydimethylsiloxyethyl dimethicone, polyglyceryl-3 disiloxane dimethicone, and mixtures thereof.

In embodiments, the silicone polymer of general formula (I) is chosen from the silicone polymers sold by Shin-Etsu under the references KF 6100®, KF 6104® and KF 6105′, and mixtures thereof.

In embodiments, the cosmetic compositions combine polyglyceryl-3 polydimethylsiloxyethyl dimethicone and, as film-forming agent, the acrylates/stearyl acrylate/dimethicone methacrylate copolymer, such as that sold under the reference KP 561® by Shin-Etsu.

In embodiments, the cosmetic compositions combine polyglyceryl-3 polydimethylsiloxyethyl dimethicone and at least one wax chosen from polyethylene waxes, candelilla wax, hydrogenated coco-glyceride wax, and mixtures thereof.

It is apparent to one skilled in the art that certain components described above as one class of compounds can also belong to other classes of compounds. Thus, this disclosure encompasses adjusting the amount of a compound belonging to multiple classes to achieve the desired effects corresponding to each of the multiple classes.

In embodiments, the cosmetic compositions can be provided in the form of a product for making up the lips. In embodiments, the cosmetic compositions can be provided in the form of a lipstick or a lip balm.

### EXAMPLE 1

### Lipstick

A lipstick comprising the polyglyceryl-3 polydimethylsiloxyethyl dimethicone (sold under the reference KF 6104® by Shin-Etsu) was prepared, and had the composition specified in Table I.

TABLE I

| | Percentages by |
|---|---|
| | weight |
| Hydrogenated polyisobutene (Parleam HV from NOF) | 4 |
| Isononyl isononanoate | 12 |
| 2-Octyldodecanol | 4.5 |
| Diisostearyl malate | 33.9 |
| Triglycerides of 50/20/10/10 | 4 |
| lauric/palmitic/cetylic/stearic acids | |
| (Softisan 100 ® from Sasol) | |
| N-Lauroyl-L-lysine | 1 |
| Acrylates/stearyl acrylate/dimethicone methacrylate | 4 |
| copolymer (KP 561 P ® from Shin-Etsu) | |
| Dimethicone 6 cSt (KF 96) | 4 |
| Polyglyceryl-3 polydimethylsiloxyethyl dimethicone | 13 |
| (KF 6104 ® from Shin-Etsu) | |
| Preservative | q.s. |
| Polyethylene wax (MW 500) | 6.6 |
| Polyethylene wax (MW 400) | 3.8 |
| Hydrophobic pyrogenic silica treated at the surface with | 2 |
| dimethylsilane (Aerosil R 972 ® from Degussa) | |
| Pigments | 7.0 |
| Simethicone (Antifoam C ® from Dow Corning) | 0.2 |
| Total | 100 |

Procedure

An oily phase was prepared by mixing isononyl isononanoate, 2-octyldodecanol, and diisostearyl malate with polyglyceryl-3 polydimethylsiloxyethyl dimethicone and dimethicone oil under hot conditions (approximately 95° C.). The oily phase thus prepared was stirred at approximately 95° C. and the fillers (N-lauroyl-L-lysine and pyrogenic silica) were added to the mixture.

The waxes, the pigments (in the form of a pigment paste), the hydrogenated polyisobutene, and the simethicone were then added to the mixture.

The mixture thus obtained was subsequently cast in a lipstick mould and left to cool until a solid composition was obtained.

The hold, the comfort and the gloss of this composition were then measured according to the protocols described above.

The hold and the gloss of the following two commercial products were also measured according to the same methods:
Control A: Jelly Plumpy, sold by Maybelline, comprising phenyl trimethicone as a silicone polymer,
Control B: Aube Rouge Glacé, sold by Kao, comprising the glycerolated silicone with the following formula as a silicone polymer

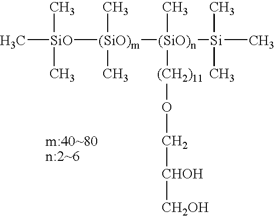

The results obtained are listed in Table II below.

TABLE II

| | Hold | Hold | | | |
|---|---|---|---|---|---|
| | towards | towards | | Index of | Mean |
| Formulation | water | oil | Transfer | comfort | gloss |
| Control A | 14.4 | 23.4 | 47.3 | | 42.5 |
| Control B | 7.0 | 8.5 | 42.0 | 85.6 | 48.2 |
| Example 1 | 5.9 | 7.9 | 32.1 | 99.5 | 48.3 |

In these measurements, the color of the inner face of the forearm is such that L*=63.9, a*=8.4, b*=13.3, and the color of the paper handkerchief is L*=97.9, a*=0.6 and b*=3.3.

The lipstick of Example 1 exhibited a better hold and a better comfort than Control A and Control B for an equivalent gloss.

In addition, the lipstick of Example 1 migrated three to four times less than Control B.

### EXAMPLES 2 and 3

### Lipstick

The percentages are by weight.

| | Example | Example |
|---|---|---|
| | 2 | 3 |
| Hydrogenated polyisobutene | | 4 |
| (Parleam HV from NOF) | | |
| Hydrogenated polyisobutene | 6 | |
| (Parleam Lite from NOF) | | |
| Isononyl isononanoate | 8 | 12 |
| 2-Octyldodecanol | 4.5 | 4.5 |
| Diisostearyl malate | 33.9 | 23.9 |
| Polyglyceryl-2 diisostearate | | 5 |
| (Cosmol 42 V from Nishin Oil) | | |
| Polyglyceryl-2 triisostearate | | 4 |
| (Cosmol 43 N from Nishin Oil) | | |
| Sorbitan sesquioleate (Cosmol | | 1 |
| 82 from Nishin Oil) | | |
| Triglycerides of 50/20/10/10 | 3 | 4 |
| lauric/palmitic/cetylic/stearic | | |
| acids (Softisan 100 ® from Sasol) | | |
| N-Lauroyl-L-lysine | 1 | 1 |
| Poly(vinyl laurate) | 3 | |
| Acrylates/stearyl acrylate/dimethicone | 2 | 4 |
| methacrylate copolymer (KP 561 P ® | | |
| from Shin-Etsu) | | |
| Dimethicone 6 cSt (KF 96) | 5 | 4 |
| Polyglyceryl-3 | 13 | 13 |
| polydimethylsiloxyethyl dimethicone | | |
| (KF6104 ® from Shin-Etsu) | | |
| Preservative | q.s. | |
| Polyethylene wax (MW 500) | 5.5 | 6.6 |
| Polyethylene wax (MW 400) | 1.1 | |
| Candelilla wax | 4.8 | |
| Microcrystalline wax | | 3.8 |
| Hydrophobic pyrogenic silica treated | 2 | 2 |
| at the surface with dimethylsilane | | |
| (Aerosil R 972 ® from Degussa) | | |
| Pigments | 7.0 | 7 |
| Simethicone (Antifoam C ® from Dow Corning) | 0.2 | 0.2 |
| Total | 100 | 100 |

Although the disclosure references various embodiments, it is to be understood that these embodiments are merely illustrative of the principles and applications of the disclosure. It is therefore to be understood that numerous modifications may be made to the embodiments and that other arrangements may be devised without departing from the spirit and scope of the disclosure, as defined by the claims.

What is claimed is:

1. An anhydrous cosmetic composition, comprising, in a physiologically acceptable medium, at least one silicone polymer of general formula (I):
R¹ₐR²_{b}R³_{c}SiO_{(4-a-b-c)/2} (I)
in which:
a is chosen from 1 to 2.5,
b and c, independently of one another, are chosen from 0.001 to 1.5,
R¹, identical or different, is chosen from:
C₁ to C₃₀ alkyl radicals, optionally substituted by one or more fluorine atoms, amino and/or carboxyl groups,
aryl or aralkyl radicals,
radicals of general formula (II):
—C_{d}H_{2d}—O—(C₂H₄O)ₑ(C₃H₆O)_{f}R⁴ (II)
with:
R⁴ being a C₁ to C₃₀ hydrocarbon radical or an R⁵—(CO)— radical with R⁵ being a C₁ to C₃₀ hydrocarbon radical,
d is an integer chosen from 0 to 15,
e and f, independently of one another, are integers chosen from 0 to 50, and
combinations thereof,
R² is a radical represented by the general formula (III):
-Q—O—X (III)
with:
Q being a divalent C₂ to C₂₀ hydrocarbon radical that optionally includes at least one ether bond and/or at least one ester bond, and
X being a polyhydroxylated hydrocarbon radical, with the proviso that, when R² is a group of general formula (IIIA):
—C₃H₆—O[CH₂CH(OH)CH₂O]ₙH (IIIA)
in which n is an integer varying from 1 to 5, then R¹ is other than a C₁₂ alkyl radical,
R³ is an organosiloxane group of general formula (IV): 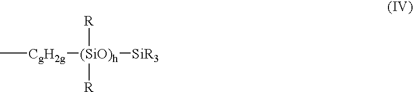
with:
each of the R radicals representing, independently of one another, a radical chosen from C₁ to C₃₀ alkyl radicals, optionally substituted by one or more fluorine atoms, and aryl and aralkyl radicals,
g is an integer chosen from 1 to 5, and
h is an integer chosen from 0 to 500.

2. An anhydrous cosmetic composition, comprising, in a physiologically acceptable medium, at least one silicone polymer of general formula (I):
R¹ₐR²_{b}R³_{c}SiO_{(4-a-b-c)/2} (I)
in which:
a is chosen from 1 to 2.5,
b and c, independently of one another, are chosen from 0.001 to 1.5,
R¹, identical or different, is chosen from:
C₁ to C₃₀ alkyl radicals, optionally substituted by one or more fluorine atoms, or amino and/or carboxyl groups,
aryl or aralkyl radicals,
radicals of general formula (II):
—C_{d}H_{2d}—O—(C₂H₄O)ₑ(C₃H₆O)_{f}R⁴ (II)
with:
R⁴ being a C₁ to C₃₀ hydrocarbon radical or an R⁵—(CO)— radical with R⁵ being a C₁ to C₃₀ hydrocarbon radical, and
d is an integer chosen from 0 to 15,
e and f, independently of one another, are integers chosen from 0 to 50, and
combinations thereof,
R² is a radical represented by the general formula (III):
-Q—O—X (III)
with:
Q being a divalent C₂ to C₂₀ hydrocarbon radical which can include at least one ether bond and/or at least one ester bond, and
X being a polyhydroxylated hydrocarbon radical,
R³ is an organosiloxane of general formula (IV): 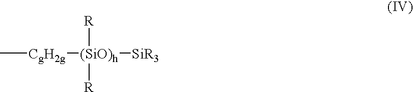
with:
each of the R radicals representing, independently of one another, a radical chosen from C₁ to C₃₀ alkyl radicals, optionally substituted by one or more fluorine atoms, and aryl or aralkyl radicals,
g is an integer chosen from 1 to 5, and
h is an integer chosen from 0 to 500,
and at least one silicone oil.

3. An anhydrous cosmetic composition, comprising, in a physiologically acceptable medium, at least one silicone polymer of general formula (I):
R¹ₐR²_{b}R³_{c}SiO_{(4-a-b-c)/2} (I)
in which:
a is chosen from 1 to 2.5,
b and c, independently of one another, are chosen from 0.001 to 1.5,
R¹, identical or different, is chosen from:
C₁ to C₃₀ alkyl radicals, optionally substituted by one or more fluorine atoms, amino and/or carboxyl groups,
aryl or aralkyl radicals,
radicals of general formula (II):
—C_{d}H_{2d}—O—(C₂H₄O)ₑ(C₃H₆O)_{f}R⁴ (II)
with:
R⁴ being a C₁ to C₃₀ hydrocarbon radical or an R⁵—(CO)— radical with R⁵ being a C₁ to C₃₀ hydrocarbon radical, and
d is an integer chosen from 0 to 15, and
e and f, independently of one another, are integers chosen from 0 to 50,
combinations thereof,
R² is a radical of general formula (III):
-Q—O—X (III)
with:
Q being a divalent C₂ to C₂₀ hydrocarbon radical which can include at least one ether bond and/or at least one ester bond,
X being a polyhydroxylated hydrocarbon radical, and
R³ is an organosiloxane of general formula (IV): 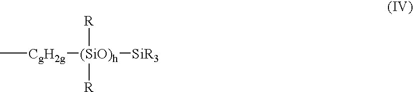
with:
each of the R radicals representing, independently of one another, a radical chosen from C₁ to C₃₀ alkyl radicals, optionally substituted by one or more fluorine atoms; and aryl or aralkyl radicals,
g is an integer chosen from 1 to 5, and
h is an integer chosen from 0 to 500,
the polymer being in a sufficient amount in the composition such that a transfer value of a deposited layer of the cosmetic composition, once spread over a substrate, is less than or equal to approximately 45.

4. An anhydrous cosmetic composition, comprising, in a physiologically acceptable medium, at least one silicone polymer of general formula (I):
R¹ₐR²_{b}R³_{c}SiO_{(4-a-b-c)/2} (I)
in which:
a is chosen from 1 to 2.5,
b and c, independently of one another, are chosen from 0.001 to 1.5,
R¹, identical or different, is chosen from:
C₁ to C₃₀ alkyl radicals, optionally substituted by one or more fluorine atoms, amino and/or carboxyl groups,
aryl or aralkyl radicals,
radicals of general formula (II):
—C_{d}H_{2d}—O—(C₂H₄O)ₑ(C₃H₆O)_{f}R⁴ (II)
with:
R⁴ being a C₁ to C₃₀ hydrocarbon radical or an R⁵—(CO)— radical with R⁵ being a C₁ to C₃₀ hydrocarbon radical, and
d is an integer chosen from 0 to 15,
e and f, independently of one another, are integers chosen from 0 to 50, and
combinations thereof,
R² is a radical of general formula (III):
-Q—O—X (III)
with:
Q being a divalent C₂ to C₂₀ hydrocarbon radical which can include at least one ether bond and/or at least one ester bond,
X being a polyhydroxylated hydrocarbon radical, and
R³ is an organosiloxane of general formula (IV): 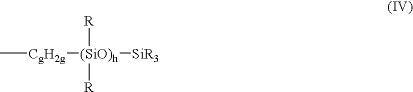
with:
each of the R radicals representing, independently of one another, a radical chosen from C₁ to C₃₀ alkyl radicals, optionally substituted by one or more fluorine atoms, and aryl or aralkyl radicals,
g is an integer chosen from 1 to 5, and
h is an integer chosen from 0 to 500,
the polymer being in a sufficient amount in the composition such that a hold of colour towards water of a deposited layer of the cosmetic composition, once spread over a substrate, is less than or equal to approximately 15.

5. An anhydrous cosmetic composition, comprising, in a physiologically acceptable medium, at least one silicone polymer of general formula (I):
R¹ₐR²_{b}R³_{c}SiO_{(4-a-b-c)/2} (I)
in which:
a is chosen from 1 to 2.5,
b and c, independently of one another, are chosen from 0.001 to 1.5,
R¹, identical or different, is chosen from:
C₁ to C₃₀ alkyl radicals, optionally substituted by one or more fluorine atoms, amino and/or carboxyl groups,
aryl or aralkyl radicals,
radicals of general formula (II):
—C_{d}H_{2d}—O—(C₂H₄O)ₑ(C₃H₆O)_{f}R⁴ (II)
with:
R⁴ being a C₁ to C₃₀ hydrocarbon radical or an R⁵—(CO)— radical with R⁵ being a C₁ to C₃₀ hydrocarbon radical, and
d is an integer chosen from 0 to 15,
e and f, independently of one another, are integers chosen from 0 to 50, and
mixtures thereof,
R² is a radical of general formula (III):
-Q—O—X (III)
with:
Q being a divalent C₂ to C₂₀ hydrocarbon radical which can include at least one ether bond and/or at least one ester bond,
X being a polyhydroxylated hydrocarbon radical, and
R³ is an organosiloxane of general formula (IV): 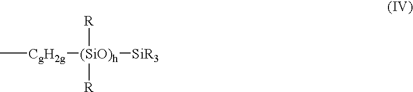
with:
each of the R radicals representing, independently of one another, a radical chosen from C₁ to C₃₀ alkyl radicals, optionally substituted by one or more fluorine atoms, and aryl or aralkyl radicals,
g is an integer chosen from 1 to 5, and
h is an integer chosen from 0 to 500,
the polymer being in a sufficient amount in the cosmetic composition such that a hold of colour towards oil of a deposited layer of the cosmetic composition, once spread over a substrate, is less than or equal to approximately 25.

6. An anhydrous cosmetic composition, comprising, in a physiologically acceptable medium, at least one silicone polymer of general formula (I):
R¹ₐR²_{b}R³_{c}SiO_{(4-a-b-c)/2} (I)
in which:
a is chosen from 1 to 2.5,
b and c, independently of one another, are chosen from 0.001 to 1.5,
R¹, identical or different, is chosen from:
C₁ to C₃₀ alkyl radicals, optionally substituted by one or more fluorine atoms, amino and/or carboxyl groups,
aryl or aralkyl radicals,
radicals of general formula (II):
—C_{d}H_{2d}—O—(C₂H₄O)ₑ(C₃H₆O)_{f}R⁴ (II)
with:
R⁴ being a C₁ to C₃₀ hydrocarbon or R⁵—(CO)— radical with R⁵ being a C₁ to C₃₀ radical,
d is an integer chosen from 0 to 15,
e and f, independently of one another, are integers chosen from 0 to 50,
and combinations thereof,
R² is a radical of general formula (III):
-Q—O—X (III)
with:
Q being a divalent C₂ to C₂₀ hydrocarbon radical which can include at least one ether bond and/or at least one ester bond, and
X being a polyhydroxy hydrocarbon radical,
R³ is an organosiloxane of general formula (IV): 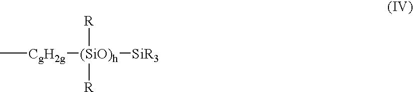
with:
each of the R groups representing, independently of one another, a radical chosen from C₁ to C₃₀ alkyl radicals, optionally substituted by one or more fluorine atoms, aryl or aralkyl radicals,
g is an integer chosen from 1 to 5, and
h is an integer chosen from 0 to 500,
the polymer being in a sufficient amount in the cosmetic composition such that a mean gloss of a deposited layer of the cosmetic composition, once spread over a substrate, is equal to or greater than approximately 30.

7. An anhydrous cosmetic composition in the cast form, comprising, in a physiologically acceptable medium, at least one silicone polymer of general formula (I):
R¹ₐR²_{b}R³_{c}SiO_{(4-a-b-c)/2} (I)
in which:
a is chosen from 1 to 2.5, and
b and c, independently of one another, are chosen from 0.001 to 1.5,
R¹, identical or different, is chosen from:
C₁ to C₃₀ alkyl radicals, optionally substituted by one or more fluorine atoms, amino and/or carboxyl groups,
aryl or aralkyl radicals,
radicals of general formula (II):
—C_{d}H_{2d}—O—(C₂H₄O)ₑ(C₃H₆O)_{f}R⁴ (II)
with:
R⁴ being a C₁ to C₃₀ hydrocarbon or R⁵—(CO)— radical with R⁵ being a C₁ to C₃₀ radical,
d is an integer chosen from 0 to 15, and
e and f, independently of one another, are integers chosen from 0 to 50,
and combinations thereof,
R² is a radical of general formula (III):
-Q—O—X (III)
with:
Q being a divalent C₂ to C₂₀ hydrocarbon radical that optionally includes at least one ether bond and/or at least one ester bond, and
X being a substituted polyhydroxy hydrocarbon radical,
R³ is an organosiloxane of general formula (IV): 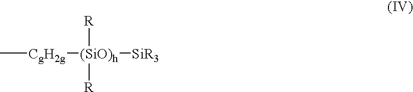
with:
each of the R groups representing, independently of one another, a radical chosen from C₁ to C₃₀ alkyl radicals, optionally substituted by one or more fluorine atoms, or aryl or aralkyl radicals,
g is an integer chosen from 1 to 5, and
h is an integer chosen from 0 to 500.

8. The cosmetic composition of claim 1, wherein the silicone polymer is present in a proportion of 0.1-40% by weight of the total weight of the cosmetic composition.

9. The cosmetic composition of claim 1, wherein the silicone polymer is present in a proportion of 0.5-30% by weight of the total weight of the cosmetic composition.

10. The cosmetic composition of claim 1, wherein the silicone polymer is present in a proportion of 1-25% by weight of the total weight of the cosmetic composition.

11. The cosmetic composition of claim 1, wherein the silicone polymer is present in a proportion of 5-20% by weight of the total weight of the cosmetic composition.

12. The cosmetic composition of claim 1, wherein the silicone polymer is present in a proportion of 7-15% by weight of the total weight of the cosmetic composition.

13. The cosmetic composition of claim 1, wherein the cosmetic composition has a transfer value of less than or equal to approximately 45.

14. The cosmetic composition of claim 1, wherein the cosmetic composition has a transfer value of less than or equal to approximately 40.

15. The cosmetic composition of claim 1, wherein the cosmetic composition has a transfer value of less than or equal to approximately 35.

16. The cosmetic composition of claim 1, wherein the cosmetic composition has a hold of colour towards water of less than or equal to approximately 15.

17. The cosmetic composition of claim 1, wherein the cosmetic composition has a hold of colour towards water of less than or equal to approximately 10.

18. The cosmetic composition of claim 1, wherein the cosmetic composition has a hold of colour towards water of less than or equal to approximately 6.

19. The cosmetic composition of claim 1, wherein the cosmetic composition has a hold of colour towards oil of less than or equal to approximately 25.

20. The cosmetic composition of claim 1, wherein the cosmetic composition has a hold of colour towards oil of less than or equal to approximately 10.

21. The cosmetic composition of claim 1, wherein the cosmetic composition has a hold of colour towards oil of less than or equal to approximately 8.

22. The cosmetic composition of claim 1, wherein the cosmetic composition has a mean gloss value of greater than or equal to approximately 30.

23. The cosmetic composition of claim 1, wherein the cosmetic composition has a mean gloss value of greater than or equal to approximately 40.

24. The cosmetic composition of claim 1, wherein the cosmetic composition has a mean gloss value of greater than or equal to approximately 45.

25. The cosmetic composition of claim 1, further comprising at least one silicone oil.

26. The cosmetic composition of claim 25, wherein a ratio of the silicone oil to the silicone polymer is less that or equal to 80:1 by weight.

27. The cosmetic composition of claim 25, wherein a ratio of the silicone oil to the silicone polymer is less that or equal to 60:1 by weight.

28. The cosmetic composition of claim 25, wherein a ratio of the silicone oil to the silicone polymer is less that or equal to 40:1 by weight.

29. The cosmetic composition of claim 25, wherein the silicone oil is selected from the group consisting of linear silicone oils, cyclic silicone oils, and mixtures thereof.

30. The cosmetic composition of claim 25, wherein the silicone oil has a viscosity of ≦8 centistokes (8×10⁻⁶ m²/s).

31. The cosmetic composition of claim 25, wherein the silicone oil is selected from the group consisting of dimethicones; octamethylcyclotetrasiloxane; decamethylcyclopentasiloxane; dodecamethylcyclohexasiloxane; heptamethylhexyltrisiloxane; heptamethyloctyltrisiloxane; hexamethyldisiloxane; octamethyltrisiloxane; decamethyltetrasiloxane; dodecamethylpentasiloxane; non-volatile polydimethylsiloxanes (PDMSs); polydimethylsiloxanes comprising pendant alkyl or alkoxy groups and/or alkyl or alkoxy groups at the ends of the silicone chain, each group having from 2 to 24 carbon atoms; phenylated silicones; phenyl trimethicones; phenyl dimethicones; phenyl(trimethylsiloxy)diphenylsiloxanes; diphenyl dimethicones; diphenyl(methyl-diphenyl)trisiloxanes; and (2-phenylethyl)trimethylsiloxysilicates.

32. The cosmetic composition of claim 1, wherein the silicone polymer is a compound of general formula (I) in which R¹ is a C₁ to C₁₀ alkyl radical.

33. The cosmetic composition of claim 1, wherein the silicone polymer is a compound of general formula (I) in which R¹ is a C₁ to C₆ alkyl radical.

34. The cosmetic composition of claim 1, wherein the silicone polymer is a compound of general formula (I) in which R¹ is a C₁ to C₄ alkyl radical.

35. The cosmetic composition of claim 1, wherein the silicone polymer is a compound of general formula (I) in which:
a is chosen from 1 to 1.4,
b and c, independently of one another, are chosen from 0.02 to 0.04,
R¹ is a C₁ to C₁₀ alkyl radical,
R² is represented by the formula (IIIA):
—C₃H₆O[CH₂CH(OH)CH₂O]ₙH (IIIA)
in which n is chosen from 1 to 5, and
R³ is represented by the formula (IVA):
—C₂H₄(CH₃)₂SiO[(CH₃)₂SiO]ₘSi(CH₃)₃ (IVA)
in which m is chosen from 3 to 9.

36. The composition of claim 1, wherein the silicone polymer is a compound of general formula (I):
R¹ₐR²_{b}R³_{c}SiO_{(4-a-b-c)/2} (I)
in which:
a is chosen from 1 to 1.4,
b and c, independently of one another, are chosen from 0.02 to 0.04,
R¹ is a methyl radical,
R² is represented by the formula (IIIA) in which n is chosen from 1 to 5, and
R³ is represented by the formula (IVA) in which m is chosen from 3 to 9.

37. The cosmetic composition of claim 1, further comprising at least one polyol selected from the group consisting of glycerol, propylene glycol, butylene glycol, pentylene glycol, hexylene glycol, dipropylene glycol, diethylene glycol, sorbitol, hydroxypropyl sorbitol, 1,2,6-hexanetriol, glycol ethers (having in particular from 3 to 16 carbon atoms), monopropylene glycol (C₁-C₄)alkyl ethers, dipropylene glycol (C₁-C₄)alkyl ethers, tripropylene glycol (C₁-C₄)alkyl ethers, or monoethylene glycol (C₁-C₄)alkyl ethers, diethylene glycol (C₁-C₄)alkyl ethers, triethylene glycol (C₁-C₄)alkyl ethers, and mixtures thereof.

38. The cosmetic composition of claim 1, wherein the cosmetic composition is in cast form and exhibits a hardness in the range of from approximately 50 to approximately 300 g.

39. The cosmetic composition of claim 1, wherein the cosmetic composition is in cast form and exhibits a hardness varying from approximately 70 to approximately 250 g.

40. The cosmetic composition of claim 1, wherein the cosmetic composition is in cast form and exhibits a hardness varying from approximately 100 to approximately 230 g.

41. The cosmetic composition of claim 1, further comprising at least one coloring material.

42. The cosmetic composition of claim 1, further comprising at least one filler.

43. The cosmetic composition of claim 1, further comprising at least one oil.

44. The cosmetic composition of claim 43, wherein the oil is a hydrocarbon oil selected from the group consisting of hydrogenated polyisobutene, isostearyl heptanoate, isononyl isononanoate, dipentaerythritol tetrahydroxystearate/tetraisostearate, 2-octyldodecanol, isotridecyl isononanoate, diisostearyl malate, and mixtures thereof.

45. The cosmetic composition of claim 1, further comprising at least one compound selected from the group consisting of waxes, pasty fatty substances, and mixtures thereof.

46. The cosmetic composition of claim 1, wherein the composition is suitable for making up and/or caring for at least one of human lips and human skin.

47. The cosmetic composition of claim 1, wherein the cosmetic composition is a lipstick.

48. A method of making the cosmetic composition of claim 1, comprising combining the at least one silicone polymer of general formula (I) and the physiologically acceptable medium.

49. The method of claim 48, comprising combining a silicone oil and the at least one silicone polymer of general formula (I).

50. The method of claim 49, wherein the silicone oil is selected from the group consisting of linear silicone oils, cyclic silicone oils, and mixtures thereof.

51. The method of claim 49, wherein the silicone oil has a viscosity of ≦8 centistokes (8×10⁻⁶ m²/s).

52. The method of claim 48, wherein the cosmetic composition has a transfer value of less than or equal to approximately 45.

53. The method of claim 48, wherein the cosmetic composition has a transfer value of less than or equal to approximately 40.

54. The method of claim 48, wherein the cosmetic composition has a transfer value of less than or equal to approximately 35.

55. The method of claim 48, wherein the cosmetic composition has a hold of colour towards water of less than or equal to approximately 15.

56. The method of claim 48, wherein the cosmetic composition has a hold of colour towards water of less than or equal to approximately 10.

57. The method of claim 48, wherein the cosmetic composition has a hold of colour towards water of less than or equal to approximately 6.

58. The method of claim 48, wherein the cosmetic composition has a hold of colour towards oil of less than or equal to approximately 25.

59. The method of claim 48, wherein the cosmetic composition has a hold of colour towards oil of less than or equal to approximately 10.

60. The method of claim 48, wherein the cosmetic composition has a hold of colour towards oil of less than or equal to approximately 8.

61. The method of claim 48, wherein the cosmetic composition has a mean gloss value of greater than or equal to approximately 30.

62. The method of claim 48, wherein the cosmetic composition has a mean gloss value of greater than or equal to approximately 40.

63. The method of claim 48, wherein the cosmetic composition has a mean gloss value of greater than or equal to approximately 45.

64. The method of claim 48, further comprising molding the cosmetic composition in cast form.
